(19) United States
(12) Patent Application Publication
(10) Pub. No.: US 2024/0156938 A1
(43) Pub. Date: May 16, 2024

(54) COMPOSITIONS AND METHODS FOR TREATING AND PREVENTING HUMAN CHLAMYDIAL INFECTIONS AND DISEASES USING ATTENUATED ANIMAL CHLAMYDIA

(71) Applicant: Board of Regents, The University of Texas System, AUSTIN, TX (US)

(72) Inventor: GUANGMING ZHONG, San Antonio, TX (US)

(21) Appl. No.: 18/506,636

(22) Filed: Nov. 10, 2023

Related U.S. Application Data
(60) Provisional application No. 63/424,281, filed on Nov. 10, 2022.

(51) Int. Cl. A61K 39/118; A61K 9/00; A61P 31/04; C07K 14/005; C12N 1/20

(52) U.S. Cl. CPC A61K 39/118; A61K 9/0053; A61P 31/04; C07K 14/005; C12N 1/205; A61K 2039/523

(57) ABSTRACT

Disclosed herein, are compositions and methods using Chlamydia muridarum organisms in the treatment and prevention of human Chlamydia trachomatis infections in a subject. Also, disclosed herein are compositions and methods for eliciting an immune response in a subject and for use as vectors.

### CROSS REFERENCE TO RELATED APPLICATIONS

This application claims the benefit of the filing date of U.S. Provisional Application No. 63/424,281, filed on Nov. 10, 2022. The content of this earlier filed application is hereby incorporated by reference in its entirety.

### REFERENCE TO A SEQUENCE LISTING

The present application contains a Sequence Listing that is submitted concurrent with the filing of this application in XML format, containing the file name “21105_0092P1_SL.xml,” created on Nov. 2, 2023, and having a size of 45,056 bytes. The Sequence Listing is hereby incorporated by reference pursuant into the present application in its entirety.

### BACKGROUND

Chlamydia trachomatis is an obligate intracellular Gram-negative bacterium that is the leading cause of bacterial sexually transmitted disease worldwide. The majority of genital chlamydial infections are initially asymptomatic and untreated, despite the availability of effective antimicrobial therapy, and may lead to severe complications such as pelvic inflammatory disease, ectopic pregnancy and infertility. Additionally, the incidence rates of genital chlamydial infections have increased over the last decade, indicating the need for an effective chlamydial vaccines.

### SUMMARY

Disclosed herein are isolated Chlamydia muridarum cells comprising: a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion of M1-R22 or a deletion of M1-P33 in open reading frame TC0412, wherein the amino acid numbering is based on the amino acid sequence of SEQ ID NO: 18; and/or f) a deletion of M1 in open reading frame TC0708, wherein the amino acid numbering is based on the amino acid sequence of SEQ ID NO: 22, wherein the Chlamydia muridarum cell has a phenotype due to the mutation, deletion or substitution of a), b), c), d), e) and/or f) of attenuated pathogenicity.

Disclosed herein are polypeptides comprising a substitution at one or more of a) T13 in open reading frame TC0168, wherein the amino acid numbering is based on the amino acid sequence of SEQ ID NO: 3; and/or b) P280 in open reading frame TC0408, wherein the amino acid numbering is based on the amino acid sequence of SEQ ID NO: 14.

Disclosed herein are polypeptides comprising a deletion at Q313 in open reading frame TC0342, wherein the amino acid numbering is based on the amino acid sequence of SEQ ID NO: 10.

Disclosed herein are polypeptides comprising a mutation at L341 in open reading frame TC0341, wherein the amino acid numbering is based on the amino acid sequence of SEQ ID NO: 6.

Disclosed herein are polypeptides comprising a deletion at one or more of a) Q313 in open reading frame TC0342, wherein the amino acid numbering is based on the amino acid sequence of SEQ ID NO: 10; b) M1-R22 or M1-P33 in open reading frame TC0412, wherein the amino acid numbering is based on the amino acid sequence of SEQ ID NO: 20; and/or c) M1 in open reading frame TC0708, wherein the amino acid numbering is based on the amino acid sequence of SEQ ID NO: 22.

Other features and advantages of the present compositions and methods are illustrated in the description below, the drawings, and the claims.

### BRIEF DESCRIPTION OF THE DRAWINGS

FIG. 1 shows that oral inoculation with a Cm (Chlamydia muridarum) G13 variant induces protection against Ct (Chlamydia trachomatis) infection in the genital tract.

FIG. 2 shows that oral inoculation with a Cm (Chlamydia muridarum) G13 variant induces protection against Ct (Chlamydia trachomatis) pathogenicity in the genital tract.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Disclosed herein are oral vaccines for protecting humans against mucosal infections and pathology caused by Chlamydia trachomatis (CT). Disclosed herein are oral inoculations for inducing immunity against the heterologous chlamydial species. The Chlamydia muridarum (CM) genome is larger than that of the CT genome, covering most CT open reading frames (ORFs).

The compositions and methods disclosed herein can have advantages over current available compositions and methods, including but not limited to, the following: 1) a single dose via the oral route is convenient and efficacious; 2) because the CM genome is 20% larger than that of the CT genome, and, thus, covering most CT ORFs with more complete amino acid sequences for many ORFs, CM ORFs may be more immunogenic and CM-induced immunity can cross-react with most CT ORFs; 3) CM is not associated with any known human diseases despite the extensive existence in natural environment and laboratory usages; and 4) CM can be more invasive at the tissue level, which may increase its immunogenicity.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

The term “subject” further includes any animal susceptible to infection by a Chlamydia species (e.g., Chlamydia trachomatis or Chlamydia muridarum). Such a subject can be a mammal (e.g., a laboratory animal such as a rat, mouse, guinea pig, rabbit, primates, etc.), a farm or commercial animal (e.g., a cow, horse, goat, donkey, sheep, etc.), a domestic animal (e.g., cat, dog, ferret, etc.), an avian species and in particular aspects, is a human. A “subject in need thereof” is a subject known to be, or suspected of being, infected with, or at risk of being infected with, Chlamydia. A subject can also include a subject not previously known or suspected to be infected by Chlamydia or in need of treatment for Chlamydia infection. For example, a subject can be administered the compositions disclosed herein even if it is not known or suspected that the subject is infected with Chlamydia (e.g., prophylactically). In some aspects, a subject can be known or believed to be at risk of infection by Chlamydia.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

As used herein, the term “patient” refers to a subject afflicted with a disease or disorder. The term “patient” includes human and veterinary subjects. In some aspects of the disclosed methods, the “patient” has been diagnosed with a need for treatment for or prevention of an infection, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be an infection or associated with an infection.

In some aspects, “treat” is meant to mean administer a compound, composition or molecule of the invention to a subject, such as a human or other mammal (for example, an animal model), that has an infection, in order to prevent or delay a worsening of the effects of the disease or condition, or to partially or fully reverse the effects of the disease.

In some aspects, the terms “treat,” “treating,” or “treatment” refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as would be well known in the art.

As used herein, the term “ameliorate” refers to the ability to make better, or more tolerable, a condition such as a chlamydial infection or a disorder associated with a chlamydial infection.

As used herein, the phrases “effective response” or “responding effectively” means a positive or beneficial response to a particular treatment in contrast to a “lack of an effective response” which can be an ineffectual, negative or detrimental response as well as the lack of a positive or beneficial response. An effective response or lack of effective response (i.e., ineffective response) is detected by evaluation, according to known protocols, of various immune functions (e.g., cell-mediated immunity, humoral immune response, etc.) and pharmacological and biological functions as would be known in the art.

The phrase “effective amount” refers to an amount of a composition as disclosed herein that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art.

The terms “immunogenic amount” or “effective immunizing dose,” as used herein, unless otherwise indicated, mean a dose of a composition disclosed herein that is sufficient to induce an immune response (which can be a protective response) in the treated subject that is greater than the inherent immunity of non-immunized subjects. An immunogenic amount or effective amount or effective immunizing dose in any particular context can be routinely determined using methods known in the art.

In some aspects, an effective immunizing dose or immunogenic amount or effective amount can comprise one or more (e.g., two or three or four or more) doses of the immunogenic composition of this invention at any time interval (e.g., hourly, daily, weekly, monthly, yearly, etc.) so as to achieve and/or maintain the desired level of protection and/or other therapeutic benefit.

The terms “vaccine,” “vaccination,” and “immunization” are well-understood in the art, and are used interchangeably herein. For example, the terms vaccine, vaccination, or immunization can be understood to be a process or composition that increases a subject's immune reaction to an immunogen (e.g., by providing an active immune response), and therefore its ability to resist, overcome and/or recover from infection (i.e., a protective immune response).

The terms “protective immunity” or “protective immune response,” as used herein, are intended to mean that the subject mounts an active immune response to the immunogenic composition and/or that the subject has been provided with passive immunity, such that upon subsequent exposure or a challenge, the animal is able to resist and/or overcome infection and/or disease. Thus, a protective immune response will decrease the incidence of morbidity and/or mortality from subsequent exposure to the chlamydial pathogens as described herein.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in Immunology: Basic Processes 117 (Joseph A. Bellanti ed., 1985).

Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. As used herein, “modulate” is meant to mean to alter, by increasing or decreasing.

The terms “preventing,” “blocking,” “antagonizing,” or “reversing” mean preventing in whole or in part, or ameliorating or controlling.

In some aspects, the term “prevent” is meant to mean minimize the chance that a subject who has an increased susceptibility for developing an infection will develop an infection. In some aspects, “prevent,” “preventing,” and “prevention” are used herein to include imparting any level of prevention or protection which is of some benefit to a subject, such that there is a reduction in the incidence and/or the severity of the disease in a treated subject, regardless of whether the protection or reduction in incidence and/or severity is partial or complete.

As used herein, the terms “prime,” “primed” or “priming” (and grammatical variations thereof) mean to initiate an active immune response that is less than protective until a second dose (booster) is given at a later time.

As used herein, terms “boost” or “booster” mean a second immunization, after an initial (or “priming”) immunization that enhances the immune response of the subject. In some aspects, the composition disclosed herein can produce an anamnestic response against a Chlamydia infection, in a sensitized subject, comprising an anamnestic response-inducing amount of a Chlamydia protein immunizing component. As used herein, the term “anamnestic response” means a secondary (booster) immune response in a sensitized subject. The term “sensitized subject” refers to a subject that has previously been in contact with a chlamydial antigen or antigens, either by natural exposure or by vaccination (primary immunization) with Chlamydia protein immunizing components.

As used herein, the terms “reduce,” “reduced,” “reducing,” and “reduction” (and grammatical variations thereof), refers to a decrease in a chlamydial infection- or disease-related parameter or symptom that is of some therapeutic value or benefit to the subject.

As used herein, the terms “elicit” or “induce” or “produce” (or grammatical variations thereof) in the context of an immune response against Chlamydia are intended to encompass the activation and/or stimulation of cells and other components of the immune system in a subject to ameliorate the effects of chlamydial infection in the subject. In some aspects, the immune response can be a protective immune response, for example, as desired in vaccination methods to treat and/or prevent infection. Protection is not required if there is some other purpose for inducing the immune response, for example, for research purposes or to produce antibody for passive immunizations or as a reagent (e.g., to detect, isolate and/or identify Chlamydia species).

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “fragment” can refer to a portion (e.g., at least 5, 10, 25, 50, 100, 125, 150, 200, 250, 300, 350, 400 or 500, etc. amino acids or nucleic acids) of a peptide that is substantially identical to a reference peptide and retains the biological activity of the reference peptide. In some aspects, the fragment or portion of a peptide retains at least 50%, 75%, 80%, 85%, 90%, 95% or 99% of the biological activity of the reference peptide described herein. A fragment of a referenced peptide can be a continuous or contiguous portion of the referenced polypeptide (e.g., a fragment of a reference peptide that is ten amino acids long can be any 2-9 contiguous residues within that reference peptide).

As used herein, the term “fragment” means a fragment (e.g., a peptide) of a protein that can stimulate either humoral or cellular immune responses in the subject. The fragment of peptide or protein can be an immunogenic fragment. In some aspects, the fragment or immunogenic fragment can comprise, consist essentially of and/or consist of one, two, three, four or more epitopes of a protein disclosed herein. In some aspects, the fragment or immunogenic fragment can be any fragment of contiguous amino acids of a protein disclosed herein and can be, for example, 5, 10, 15, 20, 25, 30, 35, 40, 45, 50, 75, 100, 150, 200, 250, 300, 350, 400, 450, 500 or 550 amino acids in length. Identification of any such fragments is routine in the art.

The term “variant” can refer to a peptide or gene product that displays modifications in sequence and/or functional properties (i.e., altered characteristics) when compared to the wild-type peptide or gene product. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level. In some aspects, the term “variant” can mean a difference in some way from the reference sequence other than just a simple deletion of an N- and/or C-terminal amino acid residue or residues. In some aspects, a variant can include a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gin, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the term “isolated” means the protein or polypeptide or immunogenic fragment or nucleic acid or cell as disclosed herein is sufficiently free of contaminants or cell components or other biological components with which polypeptides and/or nucleic acids and/or cells normally occur. “Isolated” does not mean that the preparation is technically pure (homogeneous), but it is sufficiently pure to provide the polypeptide or nucleic acid in a form in which it can be used therapeutically. Furthermore, an isolated cell is a cell that has been separated from other components with which it is normally associated in nature. For example, an isolated cell can be a cell in culture medium and/or a cell in a pharmaceutically acceptable carrier as disclosed herein.

As used herein, the term “about,” when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, “attenuated pathogenicity” means that infection with the Chlamydia cell of this invention results in reduced levels of hydrosalpinx and/or reduced levels of inflammatory cytokines (i.e., reduced inflammatory stimulation), relative to a Chlamydia cell lacking the substitutions, deletions, and/or mutations described herein.

As used herein, a “start-loss mutation” refers to a point mutation in a transcripts AUG start codon, resulting in the reduction or elimination of protein production. For example, a start-loss mutation can affect the initiation codon, i.e. the very first amino acid of the protein (which is a methionine), and this can have an effect on the final protein structure.

Compositions and Methods

Disclosed herein are methods and compositions that can be used to prevent or treat microbial or bacterial infections or disease associated therewith such as, for example, by a vaccination. In some aspects, the microbial or bacterial infection or disease associated therewith can be Chlamydia trachomatis.

Compositions

Disclosed herein are isolated Chlamydia muridarum cells. In some aspects, the Chlamydia muridarum cells can comprise a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion of M1-R22 or a deletion of M1-P33 in open reading frame TC0412, wherein the amino acid numbering is based on the amino acid sequence of SEQ ID NO: 18; and/or f) a deletion of M1 in open reading frame TC0708, wherein the amino acid numbering is based on the amino acid sequence of SEQ ID NO: 22., wherein the Chlamydia muridarum cell has a phenotype due to the mutation, deletion or substitution of a), b), c), d), e) and/or f) of attenuated pathogenicity.

In some aspects, the substitution at T13 in open reading frame TC0168, wherein the amino acid numbering is based on the amino acid sequence of SEQ ID NO: 3 can be the substitution T13R.

In some aspects, the mutation at L341 in open reading frame TC0341, wherein the amino acid numbering is based on the amino acid sequence of SEQ ID NO: 6 can introduce a stop codon at position 341.

In some aspects, the deletion at Q313 in open reading frame TC0342, wherein the amino acid numbering is based on the amino acid sequence of SEQ ID NO: 10 can be a frameshift mutation at Q313.

In some aspects, the substitution at P280 in open reading frame TC0408, wherein the amino acid numbering is based on the amino acid sequence of SEQ ID NO: 14 can be the substitution P280Q.

In some aspects, the a deletion of M1-R22 or a deletion of M1-P33 in open reading frame TC0412, wherein the amino acid numbering is based on the amino acid sequence of SEQ ID NO: 18 can be a start-loss mutation.

In some aspects, the a deletion of M1 in open reading frame TC0708, wherein the amino acid numbering is based on the amino acid sequence of SEQ ID NO: 22 can be a start-loss mutation.

In some aspects, the substitution in open reading frame TC0168 can be T13R wherein the amino acid numbering is based on the amino acid sequence of SEQ ID NO: 3. In some aspects, the substitution at T13 can be with any other amino acid, such that the phenotype of attenuated pathogenicity is retained. The substitutions can be conservative substitutions or non-conservative substitutions. Examples of amino acids that can be substituted at T13 are listed in Table 1 and Table 2. For example, the variant protein can correspond to the amino acid sequence of SEQ ID NO: 4.

In some aspects, the substitution in open reading frame TC0408 can be P280Q wherein the amino acid numbering is based on the amino acid sequence of SEQ ID NO: 14. In some aspects, the substitution at P280 can be with any other amino acid, such that the phenotype of attenuated pathogenicity is retained. The substitutions can be conservative substitutions or non-conservative substitutions. Examples of amino acids that can be substituted at P280 are listed in Table 1 and Table 2. For example, the variant protein can correspond to the amino acid sequence of SEQ ID NO: 15.

In some aspects, the mutation in open reading frame TC0341 can be a mutation that introduces a stop codon at position 341. In some aspects, the mutation in open reading frame TC0341 wherein the amino acid numbering is based on the amino acid sequence of SEQ ID NO: 6. In some aspects, the mutation in open reading frame TC0341 that introduces a stop codon at position 341 can result in a truncated protein. For example, the truncated protein can correspond to the amino acid sequence of SEQ ID NO: 8.

In some aspects, the deletion in open reading frame TC0342 can be a deletion that introduces a frameshift mutation at position Q313. In some aspects, the deletion in open reading frame TC0342 wherein the amino acid numbering is based on the amino acid sequence of SEQ ID NO: 10. For example, deletion in open reading frame TC0342 at position Q313 can correspond to the amino acid sequence of SEQ ID NO: 12.

In some aspects, the deletion in open reading frame TC0412 can be a deletion at M1 that can be a start-loss mutation. For example, the deletion in open reading frame TC0412 at M1 can correspond to the amino acid sequence of SEQ ID NO: 20. In some aspects, the deletion in open reading frame TC0412 at M1 can be a deletion of the first 21 amino acids, wherein the amino acid numbering is based on the amino acid sequence of SEQ ID NO: 18. In some aspects, the deletion in open reading frame TC0412 can be a deletion at M1-R22 or a deletion of M1-P33. For example, the deletion in open reading frame TC0412 can be a deletion can correspond to the amino acid sequence of SEQ ID NO: 20.

In some aspects, the deletion in open reading frame TC0708 can be a deletion at M1 that can be a start-loss mutation. For example, the deletion in open reading frame TC0708 at M1 can result in a reduction or elimination of protein being produced due to the deletion of the third G in the start codon as there is no alternative start codon, wherein the amino acid numbering is based on the amino acid sequence of SEQ ID NO: 22.

In some aspects, the isolated Chlamydia muridarum cell as described herein can further comprise a heterologous nucleic acid molecule. In some aspects, the heterologous nucleic acid molecule can encode a therapeutic protein or peptide and/or a functional RNA molecule. Disclosed herein are wild type Chlamydia muridarum sequences that have the amino acid sequences of SEQ ID NOs: 1, 5, 9, 13, 17, and 21. Also disclosed herein are variant Chlamydia muridarum sequences that have the amino acid sequences of SEQ ID NOs: 2, 7, 11, 15, 19, and 23. Table 3 shows nucleic acid and protein reference (wild type) sequences and variant Chlamydia muridarum sequences wherein the bold underline indicates a mutation. Table 4 provides mutations, and the corresponding predicted protein function affected.

Also disclosed herein are compositions comprising one or more of the isolated Chlamydia muridarum cells as described herein. In some aspects, the compositions can further comprise pharmaceutically acceptable carriers.

Further disclosed herein are isolated polypeptides comprising one or more substitutions, deletions, mutations or a combination thereof. In some aspects, the disclosed polypeptides can be used as a vaccine to stimulate an immune response in a subject.

Disclosed herein are polypeptides comprising a substitution at one or more of a) T13 in open reading frame TC0168, wherein the amino acid numbering is based on the amino acid sequence of SEQ ID NO: 3; and/or b) P280 in open reading frame TC0408, wherein the amino acid numbering is based on the amino acid sequence of SEQ ID NO: 14.

Disclosed herein are polypeptides having the amino acid sequence of SEQ ID NO: 4.

Disclosed herein are polypeptides having the amino acid sequence of SEQ ID NO: 16.

Disclosed herein are polypeptides comprising a deletion at Q313 in open reading frame TC0342, wherein the amino acid numbering is based on the amino acid sequence of SEQ ID NO: 10.

Disclosed herein are polypeptides having the amino acid sequence of SEQ ID NO: 12.

Disclosed are polypeptides comprising a mutation at L341 in open reading frame TC0341, wherein the amino acid numbering is based on the amino acid sequence of SEQ ID NO: 6.

Disclosed herein are polypeptides having the amino acid sequence of SEQ ID NO: 8.

Disclosed herein are polypeptides comprising a deletion at one or more of a) Q313 in open reading frame TC0342, wherein the amino acid numbering is based on the amino acid sequence of SEQ ID NO: 10; b) M1 in open reading frame TC0412, wherein the amino acid numbering is based on the amino acid sequence of SEQ ID NO: 20; and/or c) M1 in open reading frame TC0708, wherein the amino acid numbering is based on the amino acid sequence of SEQ ID NO: 22.

Disclosed herein are polypeptides having the amino acid sequence of SEQ ID NO: 19.

Also disclosed herein are compositions comprising any of the polypeptides described herein. In some aspects, the compositions can further comprise pharmaceutically acceptable carriers.

In some aspects, the Chlamydia cell can be Chlamydia trachomatis, Chlamydia muridarum, Chlamydia pneumonia, Chlamydia psittaci, Chlamydophila abortus and/or Chlamydia caviae. In some aspects, the cell described herein can be from any species of Chlamydia, Chlamydophila and/or Parachlamdyia.

In some aspects, the Chlamydia cells disclosed herein can be used as a vector to deliver a heterologous nucleotide sequence or heterologous nucleic acid molecule to a subject. In some aspects, the heterologous nucleic acid molecule can be the nucleic acid sequence of any of SEQ ID NOs: 2, 7, 11, 15, 19, or 23.

The terms “heterologous nucleotide sequence” and “heterologous nucleic acid molecule” can be used inter-changeably herein and refer to a sequence that is not naturally produced in the cell or is not naturally produced or present in the cell in the configuration or orientation in which it is present in the cell as a heterologous sequence. For example, a heterologous nucleotide sequence can encode a protein that can be naturally made by the cell, but the heterologous nucleotide sequence is present in the cell in a configuration that differs from the nucleotide sequence that is naturally present in the cell (e.g., the heterologous nucleotide sequence can be operably linked to a promoter and/or regulatory element(s) that are not naturally present in the cell or are not naturally present in the cell in the same configuration). In some aspects, the heterologous nucleic acid can comprise an open reading frame that encodes a polypeptide or nontranslated RNA of interest (e.g., for delivery to a subject for a therapeutic effect).

As used herein, the term “vector” refers to a cell disclosed herein that functions as a nucleic acid delivery vehicle, and which comprises a heterologous nucleic acid molecule to be delivered.

In some aspects, molecules that can be introduced into a subject via a Chlamydia cell described herein can include heterologous DNA, RNA, polypeptides, small organic molecules, metals, or any combinations thereof.

In some aspects, therapeutically useful molecules can be associated with the outside of the Chlamydia cell for transfer of the molecules into a subject. Such associated molecules can include DNA, RNA, small organic molecules, metals, carbohydrates, lipids and/or polypeptides. In some aspects, the therapeutically useful molecule can be covalently linked (i.e., conjugated or chemically coupled) to the surface of the Chlamydia cell. Methods of covalently linking molecules are known by those skilled in the art.

The Chlamydia cells disclosed herein can be uses in raising antibodies against a heterologous protein produced by a heterologous nucleic acid molecule and exposed on the cell surface. As a further alternative, an exogenous amino acid sequence can be attached to or inserted into the cell surface for antigen presentation to a cell, e.g., for administration to a subject to produce an immune response to the heterologous amino acid sequence.

In some aspects, a heterologous amino acid sequence can be attached to the surface of the Chlamydia cell described herein that functions as a targeting sequence to target the Chlamydia cell to certain cells or tissues. In some aspects, the targeting peptide or protein can be naturally occurring or, alternately, completely or partially synthetic. Exemplary targeting sequences can include but are not limited to ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., a, (3 or y)), neuropeptides and endorphins, and the like, and fragments thereof that retain the ability to target cells to their cognate receptors. Other illustrative peptides and proteins include but are not limited to substance P, keratinocyte growth factor, neuropeptide Y, gastrin releasing peptide, interleukin 2, hen egg white lysozyme, erythropoietin, gonadoliberin, corticostatin, 13-endorphin, leuenkephalin, rimorphin, a neoenkephalin, angiotensin, pneumadin, vasoactive intestinal peptide, neurotensin, motilin, and fragments thereof as described herein. Alternatively, in some aspects, the binding domain from a toxin (e.g., tetanus toxin or snake toxins, such as α-bungarotoxin, and the like) can be used as a targeting sequence.

Phage display techniques, as well as other techniques known in the art, can be used to identify peptides that recognize any cell type of interest.

In some aspects, the targeting sequence can encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC I glycoproteins, carbohydrate components found on membrane glycoproteins, including, mannose, N-acetyl-galactosamine, N-acetylglucosamine, fucose, galactose, and the like.

In some aspects, the targeting sequence can be a peptide that can be used for chemical coupling (e.g., can comprise arginine and/or lysine residues that can be chemically coupled through their R groups) to another molecule that targets entry into a cell.

Any heterologous nucleic acid molecule(s) of interest can be delivered in the cells disclosed herein. In some aspects, nucleic acid molecules of interest can include, for example, nucleic acids encoding polypeptides, including therapeutic (e.g., for medical or veterinary uses) or immunogenic (e.g., for vaccines) polypeptides.

In some aspects, the heterologous nucleic acid molecule disclosed herein encodes a protein or peptide or epitope of a pathogenic organism that can be sexually transmitted, including, but not limited to, Trichomonas (e.g., Trichomonas vaginalis); a pathogenic yeast or fungus (e.g., Candida albicans), Neisseria (e.g., N. gonorrhea), Treponema pallidum, and pathogenic viruses (e.g., herpes simplex virus (HSV), human immunodeficiency virus (HIV), human papilloma virus (HPV), and any combination thereof.

Examples of therapeutic polypeptides include, but are not limited to, cystic fibrosis transmembrane regulator protein (CFTR), dystrophin (including mini- and micro-dystrophins, see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sar-cospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, 13-globin, a-globin, spectrin, arantitrypsin, adenosine deaminase, hypoxanthine guanine phosphoribosyl transferase, 13-glucocerebrosidase, sphingomyelinase, lysosomal hexosaminidase A, branched-chain keto acid dehydrogenase, RP65 protein, cytokines (e.g., a-interferon, (3-inter-feron, interferon-y, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-a and 4, and the like), lysosomal acid α-glucosidase, α-galactosidase A, receptors (e.g., the tumor necrosis growth factora soluble receptor), S100A1, parvalbumin, adenylyl cyclase type 6, a molecule that modulates calcium handling (e.g., SERCA, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vaso-hibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof.

In some aspects, heterologous nucleic acid sequences encoding polypeptides include those encoding reporter polypeptides (e.g., an enzyme). Examples of reporter polypeptides include, but are not limited to, Green Fluorescent Protein, 13-galactosidase, alkaline phosphatase, luciferase, and chloramphenicol acetyltransferase gene.

Optionally, the heterologous nucleic acid can encode a secreted polypeptide (e.g., a polypeptide that can be a secreted polypeptide in its native state or that has been engineered to be secreted, for example, by operable association with a secretory signal sequence).

In some aspects, the heterologous nucleic acid can encode an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Examples of untranslated RNAs include but are not limited to RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S1 6E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

In some aspects, a nucleic acid sequence that can direct alternative splicing can be delivered. For example, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a vector.

In some aspects, the vector can also comprise a heterologous nucleic acid molecule that shares homology with and recombines with a locus on a chromosome in the subject to which the vector can be administered. In some aspects, this approach can be utilized, for example, to correct a genetic defect in a cell in the subject.

Disclosed herein are vectors that can express a polypeptide, e.g., for vaccination. In some aspects, the nucleic acid can encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

In some aspects, an immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope gp160 protein, gp41, gp120), the HIV or SIV matrix/capsid proteins, and the HIV or SIV gag, pol and env gene products). In some aspects, the immunogenic polypeptide can also be an arenavirus immunogen (e.g., Lassa fever virus immunogen, such as the Lassa fever virus nucleocapsid protein and the Lassa fever envelope glycoprotein), a pox-virus immunogen (e.g., a vaccinia virus immunogen, such as the vaccinia L1 or L8 gene products), a flavivirus immuno-gen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human corona-virus immunogen, such as the human coronavirus envelope glycoprotein, or a porcine transmissible gastroenteritis virus immunogen, or an avian infectious bronchitis virus immunogen). In some aspects, the immunogenic polypeptide can be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen can be expressed on the surface of the cancer cell. Exemplary cancer and tumor cell antigens are described in S. A. Rosenberg (Immunity 10:281 (1991)). Other examples of cancer and tumor antigens include, but are not limited to: BRCA1 gene product, BRCA2 gene product, gp100, tyrosinase, GAGE-1/2, BAGE, RAGE, LAGE, NY-ESO-1, CDK-4, I3-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178: 489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

In some aspects, the heterologous nucleic acid molecule(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

In some aspects, regulated expression of the heterologous nucleic acid molecule(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

In some aspects, a variety of promoter/enhancer elements can be used depending on the level and tissue-specific expression desired. The promoter/enhancer can be constitutive or inducible, depending on the pattern of expression desired. The promoter enhancer can be native or foreign and can be a natural or a synthetic sequence. By foreign, it is intended that the transcriptional initiation region is not found in the wild-type host into which the transcriptional initiation region can be introduced.

In some aspects, the promoter/enhancer elements can be native to the target cell or subject to be treated. In some aspects, the promoters/enhancer element can be native to the heterologous nucleic acid sequence. The promoter/enhancer element can be chosen so that it functions in the target cell(s) of interest. Further, in some aspects, the promoter/enhancer element can be a mammalian promoter/enhancer element. The promoter/enhancer element can be constitutive or inducible.

Inducible expression control elements can be advantageous in those applications in which it is desirable to provide regulation over expression of the heterologous nucleic acid sequence(s). In some aspects, inducible promoters/enhancer elements for gene delivery can be tissue-specific or -preferred promoter/enhancer elements, and can include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Examples of inducible promoters/enhancer elements include, but are not limited to, a Tet on/off element, a RU486-inducible promoter, an ecdysone-inducible promoter, a rapamycin-inducible promoter, and a metallothionein promoter.

In some aspects, wherein the heterologous nucleic acid sequence(s) is(are) transcribed and then translated in the target cells, specific initiation signals can be included for efficient translation of inserted protein coding sequences. These exogenous translational control sequences, which may include the ATG initiation codon and adjacent sequences, can be of a variety of origins, both natural and synthetic.

The vectors disclosed herein can be used in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject can impart some beneficial effect.

In some aspects, the vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In some aspects, the vectors disclosed herein can be employed to deliver a heterologous nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia ((3-globin), anemia (erythropoietin) and other blood disorders, Alzheimer's disease (GDF; neprilysin), multiple sclerosis ((3-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., a, 13, y], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin pro-peptide) and Becker, Gaucher disease (glucocerebrosidase), Hurler's disease (a-L-iduronidase), adenosine deaminase deficiency (adenosine deaminase), glycogen storage diseases (e.g., Fabry disease [a-galactosidase] and Pompe disease [lysosomal acid a-glucosidase]) and other metabolic disorders, congenital emphysema (a1-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., I1C), serca2a, zinc finger proteins that regulate the phospholamban gene, Barkct, 132-adrenergic receptor, 132-adrenergic receptor kinase (BARK), phosphoinositide-3 kinase (PI3 kinase), S100A1, parvalbumin, adenylyl cyclase type 6, a molecule that effects G-protein coupled receptor kinase type 2 knock-down such as a truncated constitutively active bARK ct; calsarcin, RNAi against phospholamban; phospholamban inhibitory or dominant-negative molecules such as phospholamban 516E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxy-lase), autoimmune diseases, and the like. The compositions and methods disclosed herein can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In some aspects, the compositions and methods disclosed herein can also be used to produce induced pluripotent stem cells (iPS). For example, a vector disclosed herein can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stem cells are known in the art. Non-limiting examples of such factors associated with stem cells and pluripotency include Oct-3/4, the SOX family (e.g., SOX1, SOX2, SOX3 and/or SOX15), the Klf family (e.g., Klf1, Klf2, Klf4 and/or Klf5), the Myc family (e.g., C-myc, L-myc and/or N-myc), NANOG and/or LIN28.

In some aspects, the vectors can be administered to a subject to elicit an immunogenic response against an immunogenic polypeptide encoded by a heterologous nucleotide sequence in the vector cell. Typically, a quantity of cells producing an immunogenically effective amount of the polypeptide in combination with a pharmaceutically acceptable carrier is administered. An “immunogenically effective amount” can be an amount of the immunogenic polypeptide that is sufficient to evoke an active immune response against the polypeptide in the subject to which the pharmaceutical formulation is administered. In some aspects, the dosage can be sufficient to produce a protective immune response. The degree of protection conferred need not be complete or permanent, as long as the benefits of administering the immunogenic polypeptide outweigh any disadvantages thereof.

The vectors disclosed herein can further be administered to elicit an immunogenic response (e.g., as a vaccine). Typically, compositions disclosed herein comprise an immunogenically effective amount of vector in combination with a pharmaceutically acceptable carrier. Optionally, the dosage can be sufficient to produce a protective immune response. The degree of protection conferred need not be complete or permanent, as long as the benefits of administering the immunogenic polypeptide outweigh any disadvantages thereof.

Disclosed herein are fragments or variants of any of the peptides, polypeptides, proteins or nucleic acids described herein. As used herein, a “fragment” refers to a peptide that is less than the full-length sequence of SEQ ID NOs: 4, 8, 12, 16, or 20. In some aspects, the peptide can be a biologically active variant of any SEQ ID NOs: 3, 4, 6, 8, 10, 12, 14, 18, 20, 22 or a fragment thereof.

Additionally, fragments or variants of any of the peptides, polypeptides or proteins described herein or known to one of ordinary skill in the art is one that retains biological function of SEQ ID NOs: 4, 8, 12, 16, or 20. A biologically active variant of a SEQ ID NOs: 3, 4, 6, 8, 10, 12, 14, 18, 20, 22 or fragment thereof is capable of eliciting an immune response to Chlamydia. Upon administration to a subject, a biologically active variant or fragment of SEQ ID NOs: 3, 4, 6, 8, 10, 12, 14, 18, 20, or 22 can elicit an immune response sufficiently at a useful affinity. As described herein, a fragment or biologically active variant thereof of SEQ ID NOs: 3, 4, 6, 8, 10, 12, 14, 18, 20, 22 or fragment thereof can be derived from any species. In some aspects, the sequences, or biologically active variants thereof, derived from one species may be identical to those derived from another species.

Where a biologically active fragment of any of SEQ ID NOs: 3, 4, 6, 8, 10, 12, 14, 18, 20, 22 is used, the fragment can be at least or about 80% identical (e.g., at least or about, 85%, 90%, 95%, 98%, 99% or 100% identical) to a corresponding variant of any of SEQ ID NOs: 4, 8, 12, 16, or 20 or wild type fragment of any of SEQ ID NOs: 3, 6, 10, 14, 18, or 22. Where a biologically active fragment of any of SEQ ID NOs: 4, 8, 12, 16, or 20 is used, the fragment can be at least or about 80% identical (e.g., at least or about, 85%, 90%, 95%, 98%, or 99% identical) to a corresponding fragment of any of SEQ ID NOs: 4, 8, 12, 16, or 20 disclosed herein. In some aspects, the biologically active fragment of any of SEQ ID NOs: 4, 8, 12, 16, or 20 can be at least or 5% identical (e.g., at least or about, 10%, 20%, 30%, 40%, 50%, 60%, 70%, 80%, 90%, 95%, 98%, or 99% identical) to a corresponding wild type amino acid sequence of any of SEQ ID NOs: 3, 6, 10, 14, 18, or 22.

In some aspects, a fragment can have an amino acid sequence of at least 90% (e.g., at least 90%, 95%, 98%, 99% or 100% identical) sequence identity when compared to a wild type fragment of any of SEQ ID NOs: 3, 6, 10, 14, 18, or 22 or a variant fragment of any of SEQ ID NOs: 4, 8, 12, 16, or 20. In some aspects, the wild type fragment of the peptide can be derived from a mouse.

The peptides disclosed herein may also include variants. Generally, the amino acid identity between an individual variant of one or more of the peptides or fragments disclosed herein can be at least 90%, 91%, 92%, 93%, 94%, 95%, 96%, 97%, 98%, 99%, or 100%. Thus, a “variant peptide” is one with the specified identity to the parent or reference peptide or fragment thereof of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent or reference peptide or fragment thereof. For example, a “variant peptide” can be a sequence that contains 1, 2, 3, 4, or more amino acid changes as compared to the parent or reference peptide or fragment thereof of the invention, and shares or improves biological function, specificity and/or activity of the parent peptide or reference peptide or fragment thereof.

In some aspects, the variant can have an amino acid sequence of at least 90% (e.g., at least 90%, 95%, 98%, 99% or 100% identical) sequence identity when compared to a reference or parent sequence. In some aspects, the reference or parent sequence can be a wild type fragment of any of SEQ ID NOs: 3, 6, 10, 14, 18, or 22. In some aspects, the wild type fragment of the peptide can be derived from a mouse.

In some aspects, any of the peptide variant sequences disclosed herein can include a single amino acid change as compared to the parent or reference peptide or fragment thereof. In some aspects, any of the peptide variant sequences disclosed herein can include at least two amino acid changes as compared to the parent or reference peptide or fragment thereof. In some aspects, the amino acid change can be a substitution, a deletion, a mutation or a combination thereof. In some aspects, the amino acid change(s) can lead to a frameshift, unfinished protein products, truncated proteins or causes a protein to terminate or end its translation earlier. The amino acid identity between individual variants can be at least 60%, 65%, 70%, 75%, 80%, 85%, 90%, 91%, 92%, 93%, 94%, 95%, 96%, 97%, 98%, 99% or 100%. Thus, an “peptide variant” can be one with the specified identity to the parent or reference peptide or fragment thereof of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent or reference peptide or fragment thereof. For example, the parent sequence can be one or more of SEQ ID NOs: 3, 4, 6, 8, 10, 12, 14, 18, 20, or 22. The variant sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 3, 4, 6, 8, 10, 12, 14, 18, 20, or 22. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent or reference peptide or fragment thereof.

As discussed herein, variant proteins or peptides are contemplated. Protein and peptide fragments, variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the peptide sequence. Typically, no more than about from 2 to 6 residues are deleted at any one site within the peptide. In some cases, more than 6 residues can be deleted at any one site within the peptide. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Substitutional variants are those in which at least one residue has been removed and a different residue inserted in its place. Such substitutions generally are made in accordance with the following Tables 1 and 2 and are referred to as conservative substitutions.

Further disclosed herein are isolated polypeptides, peptides, proteins and/or fragments that are substantially equivalent to those described for this invention. As used herein, “substantially equivalent” can refer both to nucleic acid and amino acid sequences, for example a mutant sequence, that varies from a reference sequence by one or more substitutions (e.g., substitution with conservative amino acids as are well known in the art), deletions and/or additions, the net effect of which does not result in an undesirable adverse functional dissimilarity between reference and subject sequences. In some aspects, the peptides disclosed herein can include substantially equivalent sequences that have an adverse functional dissimilarity. For purposes of the present invention, sequences having equivalent biological activity and equivalent expression characteristics are considered substantially equivalent.

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, are accomplished for example by deleting one of the basic residues or substituting one by glutaminyl or histidyl residues.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations (Rizo and Gierasch Ann. Rev. Biochem. 61:387 (1992), incorporated herein by reference).

The degree of identity can vary and can be determined by methods well established in the art. “Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the—same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. A biologically active variant or a fragment of a peptide or polypeptide described herein can have at least or about 10%, 20%, 30%, 40%, 50%, 60%, 70%, 80%, 90%, 95%, 98%, or 99% identity or homology to a corresponding naturally occurring peptide or polypeptide.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48, 443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403¬410, (1990) and Karlin et al., Proc. Natl. Acad. Sci. USA 90, 5873-5787 (1993). A particularly useful BLAST program is the WU-BLAST-2 program which was obtained from Alts¬chul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

Also disclosed herein are homologues, as well as methods of obtaining homologues, of the polypeptides and/or fragments disclosed herein from other strains of Chlamydia and/or other organisms. As used herein, an amino acid sequence or protein can be defined as a homologue of a polypeptide or fragment as disclosed herein if it shares significant homology to one of the polypeptides and/or fragments as disclosed herein. Significant homology means at least 75%, 80%, 85%, 90%, 95%, 98% and/or 100% homology with another amino acid sequence. Specifically, by using the nucleic acids that encode the chlamydial proteins and fragments as disclosed herein, as a probe or primer, and techniques such as PCR amplification and colony/plaque hybridization, one skilled in the art can identify homologues of the polypeptides and/or fragments described herein in Chlamydia and/or other organisms on the basis of information available in the art. As one non-limiting example, a listing of Chlamydia pneumoniae proteins and the Chlamydia trachomatis homologues of these proteins can be found in U.S. Pat. No. 6,822,071, the entire contents of which are incorporated by reference herein for these teachings.

As used herein, the fragments can vary in length and can be or can include contiguous amino acid residues that naturally occur in peptide wild type sequences disclosed herein or that vary to a certain degree from a naturally occurring sequence (but retain a biological activity).

Where the peptide or fragments include, at their N-terminus or C-terminus (or both), amino acid residues that are not naturally found in any of the wild type sequences disclosed herein, the additional sequence(s) can be about 1 to 200 amino acid residues long, and these residues can be divided evenly or unevenly between the N- and C-termini. For example, both the N- and C-termini can include about 1, 10, 20, 30, 40, 50, 60, 70, 80, 90, or 100 amino acid residues. Alternatively, one terminus can include about 1, 10, 20, 30, 40, 50, 60, 70, 80, 90, 100, 110, 120, 130, 140, 150, 160, 170, 180, 190, or 200 residues, and one terminus can include none.

More specifically, the N- or C-termini can include 1 to about 100 (e.g., 1, 2, 3, 4, 5, 6, 7, 8, 9, 10, 15, 20, 25, 30, 40, 50, 60, 70, 80, 90, or 100) amino acid residues that are positively charged (e.g., basic amino acid residues such as arginine, histidine, and/or lysine residues); 1 to about 100 amino acid residues that are negatively charged (e.g., acidic amino acid residues such as aspartic acid or glutamic acid residues); 1 to about 100 glycine residues; 1 to about 100 hydrophobic amino acid residues (e.g., hydrophobic aliphatic residues such as alanine, leucine, isoleucine or valine or hydrophobic aromatic residues such as phenylalanine, tryptophan or tyrosine); or 1 to about 100 (e.g., 1-4) cysteine residues. Where biologically active variants of an fragment are used, the variant can vary by substitution of one or more amino acid residues within these groups. The variants can include a conservative amino acid substitution.

The peptide variants and peptide fragments disclosed herein, including any modified fragments as well as the peptide variants disclosed herein, can be protease resistant and can include one or more types of protecting groups such as an acyl group, an amide group, a benzyl or benzoyl group, or a polyethylene glycol (PEG).

The peptide variants and fragments thereof as well as biologically active variants thereof can be modified in numerous ways. For example, agents, including additional amino acid residues, other substituents, and protecting groups can be added to either the amino terminus, the carboxy terminus, or both. The modification can be made for the purpose of altering the fragments' form or altering the way the fragments bind to or interact with other peptides or polypeptides. For example, the fragments can be modified to include cysteine residues or other sulphur-containing residues or agents that can participate in disulphide bond formation. For example, one can add at least one cysteine residue, one of which are, optionally, at the C-terminal or N-terminal of the fragment.

In some aspects, the fragments and/or polypeptides disclosed herein can be fused with a “carrier” protein or peptide to produce a fusion protein. For example, the carrier protein or peptide can be fused to a polypeptide and/or fragment described herein to increase the stability thereof (e.g., decrease the turnover rate) in the cell and/or subject. Exemplary carrier proteins include, but are not limited to, glutathione-S-transferase or maltose-binding protein. The carrier protein or peptide can alternatively be a reporter protein. For example, the fusion protein can comprise a polypeptide and/or fragment described herein and a reporter protein or peptide (e.g., green fluorescent protein (GFP), 13-glucoronidase, (3-galactosidase, luciferase, and the like) for easy detection. As a further alternative, the fusion protein attached to the polypeptides and/or fragments and a carrier protein or peptide can be targeted to a subcellular compartment of interest, i.e., to affect the co-localization of the polypeptide and/or fragment. Any suitable carrier protein as is well known in the art can be used to produce a fusion protein of this invention.

In some aspects, the cells described herein can be modified using known methods and/or administered in an adjuvant to increase immunogenicity. Methods of increasing the antigenicity or immunogenicity of a protein or peptide (e.g., on the cell surface and/or produced by the cell) are well-known in the art. The immunogenicity of the cell can also be increased through the inclusion of one or more adjuvants in addition to the cell described herein. The adjuvant can be administered with the cell, before administration of the cell, after administration of the cell, or any combination thereof.

The compositions used in the methods disclosed herein can comprise, consist essentially of and/or consist of a Chlamydia muridarum cell as described herein either alone or in combination with a chlamydial protein and/or immunogenic fragment and or epitope thereof, as well as nucleic acids encoding the chlamydial protein and/or immunogenic fragment and/or epitope thereof and can further comprise, consist essentially of and/or consist of an adjuvant.

In some aspects, the disclosed compositions can further comprise one or more than one adjuvant in the form of an amino acid sequence, and/or in the form or a nucleic acid encoding an adjuvant. The adjuvant, in the form of an amino acid sequence, can be a component of a chlamydial cell described herein and/or a separate component of the composition comprising one or more chlamydial polypeptides and/or fragments and/or epitopes thereof. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s). In some aspects, an adjuvant can be an amino acid sequence that can be a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with the cells and/or compositions disclosed herein to enhance, improve or otherwise modulate an immune response in a subject without deleterious effect on the subject.

In some aspects, an adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but can also be a salt of calcium, iron or zinc, and/or can be an insoluble suspension of acylated tyrosine, or acylated sugars, cationically or anionically derivatized polysaccharides, or polyphosphazenes.

In some aspects, the adjuvant can be MF 59, LT-K63, LT-R72 (Pal et al., Vaccine 24(6): 766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

In some aspects, the adjuvants can include, for example, a combination of monophosphoryl lipid A, preferably 3-de-0-acylated monophosphoryl, lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, a nucleic acid molecule disclosed herein can include an adjuvant by comprising a nucleotide sequence encoding an antigen of this invention and a nucleotide sequence that provides an adjuvant function, such as CpG sequences.

In some aspects, an adjuvant as described herein, such as, for example, an immunostimulatory cytokine, can be administered before, concurrent with, and/or within a few hours, several hours, and/or 1, 2, 3, 4, 5, 6, 7, 8, 9, and/or 10 days before and/or after the administration of an immunogenic chlamydial composition and/or cell of this invention to a subject.

In some aspects, any combination of adjuvants, such as immunostimulatory cytokines, can be co-administered to the subject before, after and/or concurrent with the administration of a chlamydial composition and/or cell as described herein. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition disclosed herein to a subject with and without the adjuvant or combination of adjuvants, using standard procedures, as described herein and as known in the art.

In some aspects, an immune response elicited or produced by carrying out the methods of described herein can be a protective immune response, a cellular immune response, a humoral immune response, a Th1 immune response, a Th2 immune response and any combination thereof.

To stimulate the humoral arm of the immune system, i.e., the production of antigen-specific antibodies, an immunogenic fragment can include at least about 5-10 contiguous amino acid residues of the full-length molecule, or at least about 15-25 contiguous amino acid residues of the full-length molecule, or at least about 20-50 or more contiguous amino acid residues of the full-length molecule, that define one or more epitopes, or any integer between five amino acids and the full-length sequence, provided that the fragment in question retains immunogenic activity, as measured by any art-known assay, such as, e.g., the ones described herein and/or those known in the art.

Regions of a given polypeptide that include an epitope can be identified using any number of epitope mapping techniques, well known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, each incorporated herein by reference in their entireties.

Similarly, conformational epitopes are readily identified by determining spatial conformation of amino acids such as by, e.g., x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes that are involved in stimulating the cellular arm of a subject's immune system are short peptides of about 8-25 amino acids, and these are not typically predicted by the above-described methods for identifying humoral epitopes. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenic fragments can also be identified based on their ability to stimulate lymphocyte proliferation in response to stimulation by various fragments from the antigen of interest.

The term “epitope” as used herein refers to at least about 3 to about 5, or about 5 to about 10 or about 5 to about 15, and not more than about 100, 500 or 1,000 amino acids (or any integer therebetween), which define a sequence that by itself or as part of a larger sequence, binds to an antibody generated in response to such sequence and/or stimulates a cellular immune response. There is no particular upper limit to the length of the fragment, which can comprise nearly the full-length of the protein sequence, or even a fusion protein comprising two or more epitopes from a single or multiple chlamydial proteins. An epitope for use in the present invention is not limited to a polypeptide having the exact sequence of the portion of the parent protein from which it is derived. Indeed, there are many known strains or isolates of Chlamydia and there are several variable domains that exhibit relatively high degrees of variability between isolates. Thus, the term “epitope” encompasses sequences identical to the native sequence, as well as modifications to the native sequence, such as deletions, additions and substitutions (generally, but not always, conservative in nature) that are readily produced and/or identified as epitopes according to methods standard in the art.

The terms “polypeptide” or “protein” can be used to describe a chain of amino acids that correspond to those encoded by a nucleic acid. A polypeptide or protein can be a peptide, which usually describes a chain of amino acids of from two to about 30 amino acids. The term polypeptide as used herein also describes a chain of amino acids having more than 30 amino acids and can be a fragment or domain of a protein or a full-length protein. In some aspects, the term polypeptide can refer to a linear chain of amino acids or it can refer to a chain of amino acids that has been processed and folded into a functional protein. It is understood, however, that 30 is an arbitrary number with regard to distinguishing peptides and polypeptides and the terms can be used interchangeably for a chain of amino acids. The polypeptides described herein can be obtained by isolation and purification of the polypeptides from cells where they are produced naturally, by enzymatic (e.g., proteolytic) cleavage, and/or recombinantly by expression of nucleic acid encoding the polypeptides or fragments disclosed herein. The polypeptides and/or fragments disclosed herein can also be obtained by chemical synthesis or other known protocols for producing polypeptides and fragments.

The amino acid sequences described herein are presented in the amino to carboxy direction, from left to right. Nucleotide sequences are presented herein, in the 5′ to 3′ direction, from left to right. The nucleic acids disclosed herein can be either single or double stranded (i.e., including the complementary nucleic acid). In some aspects, a nucleic acid can be the complement (e.g., complementary to the full length or only to a portion) of a nucleic acid described herein.

A “biologically active fragment” includes a polypeptide described herein that can comprise a sufficient number of amino acids to have one or more of the biological activities of the polypeptides disclosed herein. Such biological activities can include, but are not limited to, in any combination, binding activity and/or immunogenic activity, as well as any other activity now known or later identified for the polypeptides and/or fragments of this invention.

A fragment of a polypeptide or protein disclosed herein can be produced by methods well known and routine in the art. In some aspects, fragments can be produced, for example, by enzymatic or other cleavage of naturally occurring peptides or polypeptides or by synthetic protocols that are well known. Such fragments can be tested for one or more of the biological activities (e.g., immunogenicity) according to the methods described herein, which are routine methods for testing activities of polypeptides, and/or according to any art-known and routine methods for identifying such activities.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acid | pGlu |
| serine | SerS |
| threonine | ThrT |
| tyrosine | TyrY |
| tryptophan | TrpW |
| valine | ValV |

TABLE 2
Amino Acid Substitutions

Original Residue Exemplary
Conservative Substitutions,
others are known in the art.
ala; ser
arg; lys, gln
asn; gln; his
asp; glu
cys; ser
gln; asn, lys
glu; asp
gly; pro
his; asn; gln
ile; leu; val
leu; ile; val
lys; arg; gln;
met; leu; ile
phe; met; leu; tyr
ser; thr
thr; ser
trp; tyr
tyr; trp; phe
val; ile; leu

Sequences. Sequences are shown in Table 3. The nucleic acid wild type sequences (e.g., reference sequences) are from NCBI reference sequence: NC_002620.2 The mutations in variant clone G13 were identified using a genome sequence of its parental clone (G0.1.1 clone, Genbank #CP009608.1).

TABLE 3
Sequences.

| SEQ ID | | |
|---|---|---|
| NO: | Sequence | Name |
| 1 | GTGAAACGCACCTATCAACCTAGTAAAAGAAAACGT | TC0168; |
| | ACAAATTCTGTGGGATTTAGAGCTCGCATGGCTACAA | AAF39043; |
| | AAAGTGGAAGAAATCTTTTAAATCGTCGTCGCCGTCA | ribosomal protein; |
| | CGGCAGACATTCCTTAATTGATCTCTAA | wild type |
| 2 | GTGAAACGCACCTATCAACCTAGTAAAAGAAAACGT | TC0168; |
| | AGAAATTCTGTGGGATTTAGAGCTCGCATGGCTACAA | ribosomal protein; |
| | AAAGTGGAAGAAATCTTTTAAATCGTCGTCGCCGTCA | mutant |
| | CGGCAGACATTCCTTAATTGATCTCTAA | |
| 3 | MKRTYQPSKRKRTNSVGFRARMATKSGRNLLNRRRRH | TC0168; |
| | GRHSLIDL | ribosomal protein; |
| | | wild type |
| 4 | MKRTYQPSKRKRRNSVGFRARMATKSGRNLLNRRRRH | TC0168; |
| | GRHSLIDL | ribosomal protein; |
| | | mutant |
| 5 | ATGCTTAGTTGTATATTCAAAGACACGATCTTCCTGT | TC0341; |
| | CCAGTTTTTTGGCCGTTTCACTGATTTGCATGACAAC | AAF73548.1; |
| | AGCTTTATGGGGAACCATTCTTTTAGTAGAAAGACGT | ABC transporter; |
| | CCCTTGTTAAGCGAAAGTCTTTCGCATGCTTGTTACC | wild type |
| | CAGGTCTTTTGATAGGGGCCCTTCTTTCCTACAAAGT | |
| | CCCTTTATTTTCAGATTCTTTGTGGATCATTATCTTGT | |
| | GCGGCTGCGCGGCTTCTATTTTGGGATGCTTATGCAT | |
| | AGCTTTTTTAGAAAAAAAACTTGCTATGCATAAAGAT | |
| | TCAGCTTTGTGTTTAATTCTCGTTTCATTTTTTGGGGT | |
| | GGGGGTTATTCTTGTTAGCTATGTCAAAGATAGCTGT | |
| | CCTCTTTTATATAACAAGATCAATGCTTACTTGTATG | |
| | GACAAGCGGCAACTTTAGGCTATGCCGAAGCTAGGC | |
| | TGGCACTCATCATTTTTTGTTTATCAGCATTCGTGCTG | |
| | TGGTGGTGGTATCGACAAATTTCTGTAGCAATTTTCG | |
| | ATAGAGAATTTGCTTATTCCTGCGGGTTAAGAACTCG | |
| | TACGGCGGAGATGATTGTTCTTGTATTCATTTCATTA | |
| | GTCATAGTAAGTGGTGTTCGTTCTGTAGGGATTTTGC | |
| | TTATTTCGGCAATGTTTGTTGCCCCACCTTTATCTGCC | |
| | AGACAACTCTCAGATAAATTAAGTTCAATACTCATTT | |
| | TATCTAGTATTTTTGGGGGAATCTGTGGGGCTTTAGG | |
| | ATGTTATTTTTCCGTAGCGTTTACCTGCCAATCTATTA | |
| | TTGAAGGGAAGGCAACGGTAATTGTCCTCCCAACTG | |
| | GTCCACTAGTAGTATTTTTTGCGGGAGTTCTTGTTTTC | |
| | TTGTGCTTAATTTTCTCATGGAAGACTGGTTGGATCA | |
| | CGCGATATATCCGTAGGAAATTATTTTTATTTTCTCGT | |
| | GATGAGGAGCATTTATTGAAGATCTTTTGGTATTTGC | |
| | AAGAACAAAATATCTATCGTGTTAGTATGTGGGACTT | |
| | TGTTCGTTCTAGAAAGTTACAGGAATATTTTGGGTCC | |
| | AAAGCATTTTCAGGATTTAGGATGTTTTGGCTATGTA | |
| | AGAAGGGATTAGTATCTTGTTTCGATCATCAATGMTT | |
| | TTTGACGGATAAAGGAGAAGCCAAAGCGGCTAAATT | |
| | AGTTCGTGCTCATAGATTATGGGAGTCTTATCTGGTC | |
| | AGTGAATTAGATTTTAACAAAAGTAAAGTACATAATT | |
| | TCGCAGAAGAGATGGAACATGTTTTGACTGATGAATT | |
| | GGATACTACTCTATCGAAGATGCTGCAAAATCCTGAT | |
| | TATGATCCACATAAGCGAGAGATTCCAAAACTAAAA | |
| | GGGAAGTAA | |
| 6 | MLSCIFKDTIFLSSFLAVSLICMTTALWGTILLVERRPLLS | TC0341; ABC |
| | ESLSHACYPGLLIGALLSKVPLFSDSLWIIILCGCAASILG | transporter; wild |
| | CLCIAFLEKKLAMHKDSALCLILVSFFGVGVILVSYVKD | type |
| | SCPLLYNKINAYLYGQAATLGYAEARLALIIFCLSAFVL | |
| | WWWYRQISVAIFDREFAYSCGLRTRTAEMIVLVFISLVI | |
| | VSGVRSVGILLISAMFVAPPLSARQLSDKLSSILILSSIFG | |
| | GICGALGCYFSVAFTCQSIIEGKATVIVLPTGPLVVFFAG | |
| | VLVFLCLIFSWKTGWITRYIRRKLFLFSRDEEHLLKIFWY | |
| | LQEQNIYRVSMWDFVRSRKLQEYFGSKAFSGFRMFWL | |
| | CKKGLVSCFDHQXFLTDKGEAKAAKLVRAHRLWESYL | |
| | VSELDFNKSKVHNFAEEMEHVLTDELDTTLSKMLQNPD | |
| | YDPHKREIPKLKGK | |
| 7 | ATGCTTAGTTGTATATTCAAAGACACGATCTTCCTGT | TC0341; |
| | CCAGTTTTTTGGCCGTTTCACTGATTTGCATGACAAC | AAF73548.1; |
| | AGCTTTATGGGGAACCATTCTTTTAGTAGAAAGACGT | ABC transporter; |
| | CCCTTGTTAAGCGAAAGTCTTTCGCATGCTTGTTACC | variant |
| | CAGGTCTTTTGATAGGGGCCCTTCTTTCCTACAAAGT | |
| | CCCTTTATTTTCAGATTCTTTGTGGATCATTATCTTGT | |
| | GCGGCTGCGCGGCTTCTATTTTGGGATGCTTATGCAT | |
| | AGCTTTTTTAGAAAAAAAACTTGCTATGCATAAAGAT | |
| | TCAGCTTTGTGTTTAATTCTCGTTTCATTTTTTGGGGT | |
| | GGGGGTTATTCTTGTTAGCTATGTCAAAGATAGCTGT | |
| | CCTCTTTTATATAACAAGATCAATGCTTACTTGTATG | |
| | GACAAGCGGCAACTTTAGGCTATGCCGAAGCTAGGC | |
| | TGGCACTCATCATTTTTTGTTTATCAGCATTCGTGCTG | |
| | TGGTGGTGGTATCGACAAATTTCTGTAGCAATTTTCG | |
| | ATAGAGAATTTGCTTATTCCTGCGGGTTAAGAACTCG | |
| | TACGGCGGAGATGATTGTTCTTGTATTCATTTCATTA | |
| | GTCATAGTAAGTGGTGTTCGTTCTGTAGGGATTTTGC | |
| | TTATTTCGGCAATGTTTGTTGCCCCACCTTTATCTGCC | |
| | AGACAACTCTCAGATAAATTAAGTTCAATACTCATTT | |
| | TATCTAGTATTTTTGGGGGAATCTGTGGGGCTTTAGG | |
| | ATGTTATTTTTCCGTAGCGTTTACCTGCCAATCTATTA | |
| | TTGAAGGGAAGGCAACGGTAATTGTCCTCCCAACTG | |
| | GTCCACTAGTAGTATTTTTTGCGGGAGTTCTTGTTTTC | |
| | TTGTGCTTAATTTTCTCATGGAAGACTGGTTGGATCA | |
| | CGCGATATATCCGTAGGAAATTATTTTTATTTTCTCGT | |
| | GATGAGGAGCATTTATTGAAGATCTTTTGGTATTTGC | |
| | AAGAACAAAATATCTATCGTGTTAGTATGTGGGACTT | |
| | TGTTCGTTCTAGAAAGTAACAGGAATATTTTGGGTCC | |
| | AAAGCATTTTCAGGATTTAGGATGTTTTGGCTATGTA | |
| | AGAAGGGATTAGTATCTTGTTTCGATCATCAATGMTT | |
| | TTTGACGGATAAAGGAGAAGCCAAAGCGGCTAAATT | |
| | AGTTCGTGCTCATAGATTATGGGAGTCTTATCTGGTC | |
| | AGTGAATTAGATTTTAACAAAAGTAAAGTACATAATT | |
| | TCGCAGAAGAGATGGAACATGTTTTGACTGATGAATT | |
| | GGATACTACTCTATCGAAGATGCTGCAAAATCCTGAT | |
| | TATGATCCACATAAGCGAGAGATTCCAAAACTAAAA | |
| | GGGAAGTAA | |
| 8 | MLSCIFKDTIFLSSFLAVSLICMTTALWGTILLVERRPLLS | TC0341; |
| | ESLSHACYPGLLIGALLSYKVPLFSDSLWIIILCGCAASIL | AAF73548.1; |
| | GCLCIAFLEKKLAMHKDSALCLILVSFFGVGVILVSYVK | ABC transporter; |
| | DSCPLLYNKINAYLYGQAATLGYAEARLALIIFCLSAFV | variant |
| | LWWWYRQISVAIFDREFAYSCGLRTRTAEMIVLVFISLV | |
| | IVSGVRSVGILLISAMFVAPPLSARQLSDKLSSILILSSIFG | |
| | GICGALGCYFSVAFTCQSIIEGKATVIVLPTGPLVVFFAG | |
| | VLVFLCLIFSWKTGWITRYIRRKLFLFSRDEEHLLKIFWY | |
| | LQEQNIYRVSMWDFVRSRK | |
| 9 | ATGTTTGCTAGTATCTCTCCGTACTACGGGGTATCATT | TC0342; |
| | TTTTGAGTTTTTTATCGTCTTTTTTTCGCGTCTTTTTTC | AAF739203; ABC |
| | TGGGAAATTGTTTTATGATCACCTGTATATCGATGAT | transporter; wild |
| | ATTCAAGTGATTGTATTCTTTGCGATAGCCGTTTCTTG | type |
| | TTCTATAATCGGAACCTTTTTAGTTCTTAAAAAAATG | |
| | GCTATGTATGCAAACGTAGTTTCCCATACCATATTAT | |
| | TTGGTTTGGTATGCGCCTGTTTGTTTACTCATCAGCTT | |
| | ACCCATTTGTCGATGCAAAATTTGACAATAGCTGCCA | |
| | TTTCAACAACTTTATTAACCGGAGCCTCTATCCATTTT | |
| | ATTCGCAATGTATTTAAAGTAGCGGAAGAGGCTAGT | |
| | ACTGCTTTAGTTTTTTCTCTTTTGTTTTCAGCCAGCTT | |
| | ATTGCTTCTTGTGTTTTTAACAAGGAATGCTCATGTCG | |
| | GGACTGAGTTAGTTATTGGAAATGCTGATGCATTAGC | |
| | TAAAACAGATATTTTCCCAGTATTTTTGGTTTTATTGA | |
| | MTAATTTGGGCGTTTCTTACTGCTTCTTCTCGAGTTTT | |
| | ATTTGTGTATCTTTTGATACCGTATTCGCGTTTTCTTT | |
| | AGGTATTCGAGTCAAATTAATTGATTATCTTATGATG | |
| | TTTTTGCTTTCTGCTTCTATTGTTGGAGCTTTCAAAGC | |
| | TGTGGGCGTGTTAATGTCATTAGCTTTTCTACTAGTCC | |
| | CAGGATTAATCGCTAAGTTGATCGCTTCCTCTGTTCA | |
| | GGAGATGATGGGATATTCCATGATTTTTGGAGTGCTT | |
| | TCAGCTTTGATTGCTCCTGCTCTCTCTAGATCGATTTT | |
| | ATCTGTTCATGGGATTGGCTTATCGACTTCAGGGTTA | |
| | GCTGTATGTCTGTTACTTGTTTTTTACATAGGGACGCT | |
| | TGCAACTGTTTTTGTCAGAAGGCATATTTTACTTCAAT | |
| | CGAAAAATTGA | |
| 10 | MFASISPYYGVSFFEFFIVFFSRLFSGKLFYDHLYIDDIQV | TC0342; ABC |
| | IVFFAIAVSCSIIGTFLVLKKMAMYANVVSHTILFGLVCA | transporter; wild |
| | CLFTHQLTHLSMQNLTIAAISTTLLTGASIHFIRNVFKVA | type |
| | EEASTALVESLLFSASLLLLVFLTRNAHVGTELVIGNAD | |
| | ALAKTDIFPVFLVLLXNLGVSYCFFSSFICVSFDTVFAFS | |
| | LGIRVKLIDYLMMFLLSASIVGAFKAVGVLMSLAFLLVP | |
| | GLIAKLIASSVQEMMGYSMIFGVLSALIAPALSRSILSVH | |
| | GIGLSTSGLAVCLLLVFYIGTLATVFVRRHILLQSKN | |
| 11 | ATGTTTGCTAGTATCTCTCCGTACTACGGGGTATCATT | TC0342; ABC |
| | TTTTGAGTTTTTTATCGTCTTTTTTTCGCGTCTTTTTTC | transporter; variant |
| | TGGGAAATTGTTTTATGATCACCTGTATATCGATGAT | |
| | ATTCAAGTGATTGTATTCTTTGCGATAGCCGTTTCTTG | |
| | TTCTATAATCGGAACCTTTTTAGTTCTTAAAAAAATG | |
| | GCTATGTATGCAAACGTAGTTTCCCATACCATATTAT | |
| | TTGGTTTGGTATGCGCCTGTTTGTTTACTCATCAGCTT | |
| | ACCCATTTGTCGATGCAAAATTTGACAATAGCTGCCA | |
| | TTTCAACAACTTTATTAACCGGAGCCTCTATCCATTTT | |
| | ATTCGCAATGTATTTAAAGTAGCGGAAGAGGCTAGT | |
| | ACTGCTTTAGTTTTTTCTCTTTTGTTTTCAGCCAGCTT | |
| | ATTGCTTCTTGTGTTTTTAACAAGGAATGCTCATGTCG | |
| | GGACTGAGTTAGTTATTGGAAATGCTGATGCATTAGC | |
| | TAAAACAGATATTTTCCCAGTATTTTTGGTTTTATTGA | |
| | MTAATTTGGGCGTTTCTTACTGCTTCTTCTCGAGTTTT | |
| | ATTTGTGTATCTTTTGATACCGTATTCGCGTTTTCTTT | |
| | AGGTATTCGAGTCAAATTAATTGATTATCTTATGATG | |
| | TTTTTGCTTTCTGCTTCTATTGTTGGAGCTTTCAAAGC | |
| | TGTGGGCGTGTTAATGTCATTAGCTTTTCTACTAGTCC | |
| | CAGGATTAATCGCTAAGTTGATCGCTTCCTCTGTTCA | |
| | GGAGATGATGGGATATTCCATGATTTTTGGAGTGCTT | |
| | TCAGCTTTGATTGCTCCTGCTCTCTCTAGATCGATTTT | |
| | ATCTGTTCATGGGATTGGCTTATCGACTTCAGGGTTA | |
| | GCTGTATGTCTGTTACTTGTTTTTTACATAGGGACGCT | |
| | TGCAACTGTTTTTGTCAGAAGGCATATTTTACTTCATC | |
| | GAAAAATTGA | |
| 12 | MFASISPYYGVSFFEFFIVFFSRLFSGKLFYDHLYIDDIQV | TC0342; ABC |
| | IVFFAIAVSCSIIGTFLVLKKMAMYANVVSHTILFGLVCA | transporter; variant |
| | CLFTHQLTHLSMQNLTIAAISTTLLTGASIHFIRNVFKVA | |
| | EEASTALVESLLFSASLLLLVFLTRNAHVGTELVIGNAD | |
| | ALAKTDIFPVFLVLLXNLGVSYCFFSSFICVSFDTVFAFS | |
| | LGIRVKLIDYLMMFLLSASIVGAFKAVGVLMSLAFLLVP | |
| | GLIAKLIASSVQEMMGYSMIFGVLSALIAPALSRSILSVH | |
| | GIGLSTSGLAVCLLLVFYIGTLATVFVRRHILLHRKI | |
| 13 | ATGAAGCGTTCTTTGTGGTACAAAAGATTTAGCTATT | TC0408; |
| | ATCTCCTTGTAGGATTACCGTTAGCTTTTTTTGTCCTA | AAF739264; |
| | CTTCCTAAATTTCTTTCCAGTGAATCCGGGAAATATC | AAF3926.2; wild |
| | TCTTTCTTTCTGTACTGAATAAAGAAACGGGGTTGCA | type |
| | ATGCGAAATTGAACAGTTACATCTATCCTGGTTTGGC | |
| | TCCCAAACAGCTAAAAAAGTGCGTATTCGTGGAATTG | |
| | ACTCTGAATCCGAGATTTTCTCTGCAGAACAAATTAT | |
| | TGTAAGTGGATCTCTTCCTCGTTTACTTCTTTACAGAT | |
| | TCCCCAAATCCCTAACGCTGACTGGATGGACTTTACA | |
| | AATCGATGAATCTTTATCGATAAATTCTCCTTCCTTAT | |
| | ATCACCTAGATCCTGGGTCCCTCCTTTCTAAAATAGA | |
| | ACGCAGCGATATTACATCCGAGTTAGGATCCATAACC | |
| | ATGAAAACAATCAAAGGATCTACGCTTTCTGTTTCAG | |
| | GATTTTATGTAAAAAAAACAGCCCAACAATTACTCAT | |
| | CCGAGCTCTTACAAAAGAAAATGATATACAGGGTTCT | |
| | GTATCTGTAGAAGGAGCTCTTTCTCCTCATTTTCTTCT | |
| | TAATGTTCAGCTTTCTTCTGTTCCCGCTTCACTTTTTA | |
| | AGCTCTTTATTGCCTCCCCTTCTATAGATAGAATCCTA | |
| | TCAACAGAGGATCTTATTAACCTAACAGCCAAGGCTC | |
| | ACCAAGAAAAGGACTCTACTCTTATTACCTTGACCAC | |
| | AGAAGGAAATCAAATAACTGCAAAACTTCGAGGTCA | |
| | TATACGTGATAATACATTTTTAATCACTCAAGGAGGA | |
| | GCCTCATCCATAGTTTTGCCACCAGCAATAACCTCAA | |
| | GTATCCTCTCTGAGTTATTAGCAGTAGAGACCTCCAT | |
| | TCGCTCAAAAGAAGCTCACCTATTTATATCTGACGCG | |
| | AAACTTCCCCTTTCTATCTCAAAATGGAATTCTTCTGA | |
| | ATTTTCATTGCAGGCTAACTTGCCTCAAATTTCTGTAG | |
| | AAACATCCGATCCTAATCTCTCTATCCAAACAGAAAA | |
| | TACAAAAATTTCGGTAAAAAAATCTGACCGTTTTACA | |
| | CTCATTCGATCTTCTTCTGCAGCAAGACTAGGAGGAG | |
| | CCTCCCCTTCTTATATTCATGGATCTCTTTCTATAGAT | |
| | AACCGTAAACACTCTGCAGAATTTCATATCCAACAAT | |
| | CTCTCTTACCACATACGTATTTACGAGCGCTTCTTCCT | |
| | AAACCTTTCGAAATAAATATCCCCCTTGAAGTCCCTT | |
| | ATTACACTTTGGATGTCAAAGGGAAATATCGTAATAC | |
| | CCAACTCTCATGCGAAGCTTTTTTAGATAACCCCTTA | |
| | TTACGCTTATCATGCACCCTTTCCGGACCTTTACAAA | |
| | CTTTGCAATTTAATGGAGAAGGAGCCTACACGTTGTC | |
| | TGAACGCTGGAAAGACCGTTTCTCTCCTTATTTTCTGC | |
| | AAATGCAAACATCATTTTCTGGGAAAATGCATTTTAC | |
| | TCAACGACATATCTTTTTCCCAAAACTATCTGTCAAA | |
| | CTCTTTGCAGGAGAAAACGAAGTTTTCATCCATGGGA | |
| | AATTTGGTAAAGCTAATGAAGAAATTAAGCCCTCCA | |
| | ATACTTCTCTTCTAGTGTATGGGAACCTTTTTTCTCTT | |
| | CCTATGGACCTCTGTTCCTCTAAATTCGCTCCGCTGCA | |
| | AATGACACATTCAAGCTTTTCCTTACATTCTGATGGA | |
| | GGGAAAGGTGTGCTTAAAGGGAACTTAAAATTATCC | |
| | ATAACCGATCCTGAATTCCCTCACTTATCGGAAACAA | |
| | AGCTCTTAATTCCCGACATTGTGATCTCATCTTTAGAT | |
| | CCTACATCCCCTTGGGATGCGGAGAATATCTCCATTC | |
| | AAGCATCTGGAGAGCTTTTACAATTGCCTGTAGATAG | |
| | GCTCATTCGATTGCAGCATAAAGATGCCTCCTTATCC | |
| | CGTTACATTGGGGAAACAGGAAAAGCCTCTTTCCAAC | |
| | TTCTTTACTCTCCAGATAAAGAGGAAACTATCTCCAT | |
| | CGCTTCAACATTTAAAACGGATGCTTTGACTGGGGAT | |
| | TTCCGCTTTGTTATGGATAGCTCGTTATCTCTGACAGA | |
| | GAACACTTATGGCTCACTACAATGGGAAGTTTCTCCG | |
| | GAGCGTTATAGCTCTTTCTTTGAAAAAGCTTCCAGCT | |
| | CCCCATCCTGCCTTCTACATCGCCCCACTACCTTCCGC | |
| | CTAGCTCTTTCTAAAATTTCCTGCTCAGACAAAAAAA | |
| | CGGGATACTCTTGTCTTTCTCTCTTGTCAGAAGGTGG | |
| | AATAGAAGGAAAACTATCCTCCACGCCTTTAGTTTTT | |
| | TATGACCATTTATCCAAAGAAAACTTCATCGTAAACA | |
| | ATATAAATGGATCTGTTCACGCACAAAATCTAAATGA | |
| | CCTAATCCAATATAAATTCACTGGAAACTGTTTAGCT | |
| | CCTAACCAAGATAATAAATCCCCTGTATCTTTTGTCA | |
| | TTGAAGGTGAAGCCAAAAATATTTTAATGAATGAAA | |
| | CAAGAAGTTTAACCCAAACAGCAACCTGGACAAACA | |
| | TTCCGACATCCTTTATCACGGGCATTGTTCCCATTTCT | |
| | CCTGGAGTTAGAGCTAAACTCACCTCCTTAGCAGGAC | |
| | CTAAAATCCATATCTCTATCTACAATGATTTTTATAA | |
| | AGGAGAAGGACCATTAACCATTAAGGTAGATTCTGA | |
| | AAATCTGACAGGTTATTTACCTCTAGAGCTTACAGAG | |
| | CATGCTATTCTGCTAAAAGAAGACCTTACAGCATCTT | |
| | TGCACATCAATGAAGAAATTAATAAGGCATTCCTTAG | |
| | AGAATTCAACCCATTGATTTCAGAGGGGGGTGCTTAT | |
| | TCAAAACATCCAGTAAGTTTACGAGTAAATCCGCAA | |
| | AATTTTTATCTGCCTATTAAACCTTACTCTTTCGAAAA | |
| | TTTTCGTATTCAATCTGCTTCCCTAGATTTCGGGAAAA | |
| | TAGAGATAGAGAATACAGGAACTATGCAAGACCTCT | |
| | TTCAATTCTTAGATGTCGAGGCGGAGCAGCAAAGAG | |
| | TGGAATCTTGGTTTACACCTATCTTTTTCTCTGTACAA | |
| | AACGGTCAAATTATCTATAAGCGCTTTGATGCTTTAA | |
| | TCGACGGACGCATACGCTTAGCTCTATGGGGAAAAA | |
| | CCGACGTTATCCGAGACAGATTAGCAATGACTCTAGG | |
| | GATTGATCCAGAACTCATTAAAAAACTGTTCCGCAAC | |
| | ACGATGCTCAAAACGAAAAATTTTTTCTTAATCAAGA | |
| | TTCGCGGCCCTATCTCGTCACCAGAAATTGACTGGTC | |
| | GTCCGCTTACGCTAGAATCGCTCTACTGAAAAGCTAC | |
| | ACGATCGCGGGTCCTCTGAACTCATTAGCAGACAAAT | |
| | TATTTTCGTCATTAGGAGAACCCACTCCTTCACAGAC | |
| | AGTGTCCCCCCTTCCATGGGAAGTTTCTGAGTCAGAG | |
| | TAA | |
| 14 | MKRSLWYKRFSYYLLVGLPLAFFVLLPKFLSSESGKYLF | TC0408; wild type |
| | LSVLNKETGLQCEIEQLHLSWFGSQTAKKVRIRGIDSES | |
| | EIFSAEQIIVSGSLPRLLLYRFPKSLTLTGWTLQIDESLSIN | |
| | SPSLYHLDPGSLLSKIERSDITSELGSITMKTIKGSTLSVS | |
| | GFYVKKTAQQLLIRALTKENDIQGSVSVEGALSPHFLLN | |
| | VQLSSVPASLFKLFIASPSIDRILSTEDLINLTAKAHQEKD | |
| | STLITLTTEGNQITAKLRGHIRDNTFLITQGGASSIVLPPA | |
| | ITSSILSELLAVETSIRSKEAHLFISDAKLPLSISKWNSSEF | |
| | SLQANLPQISVETSDPNLSIQTENTKISVKKSDRFTLIRSS | |
| | SAARLGGASPSYIHGSLSIDNRKHSAEFHIQQSLLPHTYL | |
| | RALLPKPFEINIPLEVPYYTLDVKGKYRNTQLSCEAFLD | |
| | NPLLRLSCTLSGPLQTLQFNGEGAYTLSERWKDRFSPYF | |
| | LQMQTSFSGKMHFTQRHIFFPKLSVKLFAGENEVFIHGK | |
| | FGKANEEIKPSNTSLLVYGNLFSLPMDLCSSKFAPLQMT | |
| | HSSFSLHSDGGKGVLKGNLKLSITDPEFPHLSETKLLIPD | |
| | IVISSLDPTSPWDAENISIQASGELLQLPVDRLIRLQHKD | |
| | ASLSRYIGETGKASFQLLYSPDKEETISIASTFKTDALTG | |
| | DFRFVMDSSLSLTENTYGSLQWEVSPERYSSFFEKASSS | |
| | PSCLLHRPTTFRLALSKISCSDKKTGYSCLSLLSEGGIEG | |
| | KLSSTPLVFYDHLSKENFIVNNINGSVHAQNLNDLIQYK | |
| | FTGNCLAPNQDNKSPVSFVIEGEAKNILMNETRSLTQTA | |
| | TWTNIPTSFITGIVPISPGVRAKLTSLAGPKIHISIYNDFYK | |
| | GEGPLTIKVDSENLTGYLPLELTEHAILLKEDLTASLHIN | |
| | EEINKAFLREFNPLISEGGAYSKHPVSLRVNPQNFYLPIK | |
| | PYSFENFRIQSASLDFGKIEIENTGTMQDLFQFLDVEAEQ | |
| | QRVESWFTPIFFSVQNGQIIYKRFDALIDGRIRLALWGKT | |
| | DVIRDRLAMTLGIDPELIKKLFRNTMLKTKNFFLIKIRGP | |
| | ISSPEIDWSSAYARIALLKSYTIAGPLNSLADKLFSSLGEP | |
| | TPSQTVSPLPWEVSESE | |
| 15 | ATGAAGCGTTCTTTGTGGTACAAAAGATTTAGCTATT | TC0408; variant |
| | ATCTCCTTGTAGGATTACCGTTAGCTTTTTTTGTCCTA | |
| | CTTCCTAAATTTCTTTCCAGTGAATCCGGGAAATATC | |
| | TCTTTCTTTCTGTACTGAATAAAGAAACGGGGTTGCA | |
| | ATGCGAAATTGAACAGTTACATCTATCCTGGTTTGGC | |
| | TCCCAAACAGCTAAAAAAGTGCGTATTCGTGGAATTG | |
| | ACTCTGAATCCGAGATTTTCTCTGCAGAACAAATTAT | |
| | TGTAAGTGGATCTCTTCCTCGTTTACTTCTTTACAGAT | |
| | TCCCCAAATCCCTAACGCTGACTGGATGGACTTTACA | |
| | AATCGATGAATCTTTATCGATAAATTCTCCTTCCTTAT | |
| | ATCACCTAGATCCTGGGTCCCTCCTTTCTAAAATAGA | |
| | ACGCAGCGATATTACATCCGAGTTAGGATCCATAACC | |
| | ATGAAAACAATCAAAGGATCTACGCTTTCTGTTTCAG | |
| | GATTTTATGTAAAAAAAACAGCCCAACAATTACTCAT | |
| | CCGAGCTCTTACAAAAGAAAATGATATACAGGGTTCT | |
| | GTATCTGTAGAAGGAGCTCTTTCTCCTCATTTTCTTCT | |
| | TAATGTTCAGCTTTCTTCTGTTCCCGCTTCACTTTTTA | |
| | AGCTCTTTATTGCCTCCCCTTCTATAGATAGAATCCTA | |
| | TCAACAGAGGATCTTATTAACCTAACAGCCAAGGCTC | |
| | ACCAAGAAAAGGACTCTACTCTTATTACCTTGACCAC | |
| | AGAAGGAAATCAAATAACTGCAAAACTTCGAGGTCA | |
| | TATACGTGATAATACATTTTTAATCACTCAAGGAGGA | |
| | GCCTCATCCATAGTTTTGCAACCAGCAATAACCTCAA | |
| | GTATCCTCTCTGAGTTATTAGCAGTAGAGACCTCCAT | |
| | TCGCTCAAAAGAAGCTCACCTATTTATATCTGACGCG | |
| | AAACTTCCCCTTTCTATCTCAAAATGGAATTCTTCTGA | |
| | ATTTTCATTGCAGGCTAACTTGCCTCAAATTTCTGTAG | |
| | AAACATCCGATCCTAATCTCTCTATCCAAACAGAAAA | |
| | TACAAAAATTTCGGTAAAAAAATCTGACCGTTTTACA | |
| | CTCATTCGATCTTCTTCTGCAGCAAGACTAGGAGGAG | |
| | CCTCCCCTTCTTATATTCATGGATCTCTTTCTATAGAT | |
| | AACCGTAAACACTCTGCAGAATTTCATATCCAACAAT | |
| | CTCTCTTACCACATACGTATTTACGAGCGCTTCTTCCT | |
| | AAACCTTTCGAAATAAATATCCCCCTTGAAGTCCCTT | |
| | ATTACACTTTGGATGTCAAAGGGAAATATCGTAATAC | |
| | CCAACTCTCATGCGAAGCTTTTTTAGATAACCCCTTA | |
| | TTACGCTTATCATGCACCCTTTCCGGACCTTTACAAA | |
| | CTTTGCAATTTAATGGAGAAGGAGCCTACACGTTGTC | |
| | TGAACGCTGGAAAGACCGTTTCTCTCCTTATTTTCTGC | |
| | AAATGCAAACATCATTTTCTGGGAAAATGCATTTTAC | |
| | TCAACGACATATCTTTTTCCCAAAACTATCTGTCAAA | |
| | CTCTTTGCAGGAGAAAACGAAGTTTTCATCCATGGGA | |
| | AATTTGGTAAAGCTAATGAAGAAATTAAGCCCTCCA | |
| | ATACTTCTCTTCTAGTGTATGGGAACCTTTTTTCTCTT | |
| | CCTATGGACCTCTGTTCCTCTAAATTCGCTCCGCTGCA | |
| | AATGACACATTCAAGCTTTTCCTTACATTCTGATGGA | |
| | GGGAAAGGTGTGCTTAAAGGGAACTTAAAATTATCC | |
| | ATAACCGATCCTGAATTCCCTCACTTATCGGAAACAA | |
| | AGCTCTTAATTCCCGACATTGTGATCTCATCTTTAGAT | |
| | CCTACATCCCCTTGGGATGCGGAGAATATCTCCATTC | |
| | AAGCATCTGGAGAGCTTTTACAATTGCCTGTAGATAG | |
| | GCTCATTCGATTGCAGCATAAAGATGCCTCCTTATCC | |
| | CGTTACATTGGGGAAACAGGAAAAGCCTCTTTCCAAC | |
| | TTCTTTACTCTCCAGATAAAGAGGAAACTATCTCCAT | |
| | CGCTTCAACATTTAAAACGGATGCTTTGACTGGGGAT | |
| | TTCCGCTTTGTTATGGATAGCTCGTTATCTCTGACAGA | |
| | GAACACTTATGGCTCACTACAATGGGAAGTTTCTCCG | |
| | GAGCGTTATAGCTCTTTCTTTGAAAAAGCTTCCAGCT | |
| | CCCCATCCTGCCTTCTACATCGCCCCACTACCTTCCGC | |
| | CTAGCTCTTTCTAAAATTTCCTGCTCAGACAAAAAAA | |
| | CGGGATACTCTTGTCTTTCTCTCTTGTCAGAAGGTGG | |
| | AATAGAAGGAAAACTATCCTCCACGCCTTTAGTTTTT | |
| | TATGACCATTTATCCAAAGAAAACTTCATCGTAAACA | |
| | ATATAAATGGATCTGTTCACGCACAAAATCTAAATGA | |
| | CCTAATCCAATATAAATTCACTGGAAACTGTTTAGCT | |
| | CCTAACCAAGATAATAAATCCCCTGTATCTTTTGTCA | |
| | TTGAAGGTGAAGCCAAAAATATTTTAATGAATGAAA | |
| | CAAGAAGTTTAACCCAAACAGCAACCTGGACAAACA | |
| | TTCCGACATCCTTTATCACGGGCATTGTTCCCATTTCT | |
| | CCTGGAGTTAGAGCTAAACTCACCTCCTTAGCAGGAC | |
| | CTAAAATCCATATCTCTATCTACAATGATTTTTATAA | |
| | AGGAGAAGGACCATTAACCATTAAGGTAGATTCTGA | |
| | AAATCTGACAGGTTATTTACCTCTAGAGCTTACAGAG | |
| | CATGCTATTCTGCTAAAAGAAGACCTTACAGCATCTT | |
| | TGCACATCAATGAAGAAATTAATAAGGCATTCCTTAG | |
| | AGAATTCAACCCATTGATTTCAGAGGGGGGTGCTTAT | |
| | TCAAAACATCCAGTAAGTTTACGAGTAAATCCGCAA | |
| | AATTTTTATCTGCCTATTAAACCTTACTCTTTCGAAAA | |
| | TTTTCGTATTCAATCTGCTTCCCTAGATTTCGGGAAAA | |
| | TAGAGATAGAGAATACAGGAACTATGCAAGACCTCT | |
| | TTCAATTCTTAGATGTCGAGGCGGAGCAGCAAAGAG | |
| | TGGAATCTTGGTTTACACCTATCTTTTTCTCTGTACAA | |
| | AACGGTCAAATTATCTATAAGCGCTTTGATGCTTTAA | |
| | TCGACGGACGCATACGCTTAGCTCTATGGGGAAAAA | |
| | CCGACGTTATCCGAGACAGATTAGCAATGACTCTAGG | |
| | GATTGATCCAGAACTCATTAAAAAACTGTTCCGCAAC | |
| | ACGATGCTCAAAACGAAAAATTTTTTCTTAATCAAGA | |
| | TTCGCGGCCCTATCTCGTCACCAGAAATTGACTGGTC | |
| | GTCCGCTTACGCTAGAATCGCTCTACTGAAAAGCTAC | |
| | ACGATCGCGGGTCCTCTGAACTCATTAGCAGACAAAT | |
| | TATTTTCGTCATTAGGAGAACCCACTCCTTCACAGAC | |
| | AGTGTCCCCCCTTCCATGGGAAGTTTCTGAGTCAGAG | |
| | TAA | |
| 16 | MKRSLWYKRFSYYLLVGLPLAFFVLLPKFLSSESGKYLF | TC0408; variant |
| | LSVLNKETGLQCEIEQLHLSWFGSQTAKKVRIRGIDSES | |
| | EIFSAEQIIVSGSLPRLLLYRFPKSLTLTGWTLQIDESLSIN | |
| | SPSLYHLDPGSLLSKIERSDITSELGSITMKTIKGSTLSVS | |
| | GFYVKKTAQQLLIRALTKENDIQGSVSVEGALSPHFLLN | |
| | VQLSSVPASLFKLFIASPSIDRILSTEDLINLTAKAHQEKD | |
| | STLITLTTEGNQITAKLRGHIRDNTFLITQGGASSIVLQPA | |
| | ITSSILSELLAVETSIRSKEAHLFISDAKLPLSISKWNSSEF | |
| | SLQANLPQISVETSDPNLSIQTENTKISVKKSDRFTLIRSS | |
| | SAARLGGASPSYIHGSLSIDNRKHSAEFHIQQSLLPHTYL | |
| | RALLPKPFEINIPLEVPYYTLDVKGKYRNTQLSCEAFLD | |
| | NPLLRLSCTLSGPLQTLQFNGEGAYTLSERWKDRFSPYF | |
| | LQMQTSFSGKMHFTQRHIFFPKLSVKLFAGENEVFIHGK | |
| | FGKANEEIKPSNTSLLVYGNLFSLPMDLCSSKFAPLQMT | |
| | HSSFSLHSDGGKGVLKGNLKLSITDPEFPHLSETKLLIPD | |
| | IVISSLDPTSPWDAENISIQASGELLQLPVDRLIRLQHKD | |
| | ASLSRYIGETGKASFQLLYSPDKEETISIASTFKTDALTG | |
| | DFRFVMDSSLSLTENTYGSLQWEVSPERYSSFFEKASSS | |
| | PSCLLHRPTTFRLALSKISCSDKKTGYSCLSLLSEGGIEG | |
| | KLSSTPLVFYDHLSKENFIVNNINGSVHAQNLNDLIQYK | |
| | FTGNCLAPNQDNKSPVSFVIEGEAKNILMNETRSLTQTA | |
| | TWTNIPTSFITGIVPISPGVRAKLTSLAGPKIHISIYNDFYK | |
| | GEGPLTIKVDSENLTGYLPLELTEHAILLKEDLTASLHIN | |
| | EEINKAFLREFNPLISEGGAYSKHPVSLRVNPQNFYLPIK | |
| | PYSFENFRIQSASLDFGKIEIENTGTMQDLFQFLDVEAEQ | |
| | QRVESWFTPIFFSVQNGQIIYKRFDALIDGRIRLALWGKT | |
| | DVIRDRLAMTLGIDPELIKKLFRNTMLKTKNFFLIKIRGP | |
| | ISSPEIDWSSAYARIALLKSYTIAGPLNSLADKLFSSLGEP | |
| | TPSQTVSPLPWEVSESE | |
| 17 | ATGGTAAGTTTCGATTTAAGTGTGACAACAACGAAT | TC0412; |
| | ATTGGTGCAGGATATGACGATATTCAACGTATGTTAA | AAF39268; HP; |
| | ACGGTGTGACTTGTTCTTCCGGAGGAATGGGATTATT | wild type |
| | AACTCCTTCCGCATGTTCTCCAATGAGTAGTTTCTGTT | |
| | CTTCTAACCAGCCCTATAGTGCTAGAGATTTAAAGAA | |
| | TCGCATTCACCAGTTTTGTCAGCATTCGGGTCCTATTA | |
| | CGGGATTTTATTCCTTGTACAATGAGAAGATTATGTT | |
| | TGAGGAGGCTCTTCTTGTTCCGACTGTTCTTGAAGCT | |
| | GTAGAGTCAACTTTTTGGATATCAGCTCTTTCTCGTTT | |
| | AGGAGGAGAGCGTCCTTCGACTTTTGACACGGTAATT | |
| | CTGAGCTTTTTTGTTGGGCTTATTTCTTTGGTGTGCGG | |
| | GGCGATGTTTGTTGGTATTGTTTCTAGTGCCGTGAAA | |
| | ATTTATCGTCTTATGCAGACAATGAGGCAAGCACGTA | |
| | CTTTGAATGAAAATGTGCAAAGACTATTAGCTCCACA | |
| | AGCTACGAACATGCGGTCTGCTTTTGCAAAACTTAAA | |
| | GGGATTGTTGCTTCGAAAGCGCTTGATCAAGTAGAGC | |
| | AAGGTTATCGCAAATTCAGAAATCGAATGATTACTTC | |
| | TTTTGTTGCTAATGCTCTAATTACAATAGCCTTTTGTG | |
| | CTTTATTAGCATCTGTAATTCTTTCTGCTTTCTTCATT | |
| | GGTGGAGCAAGTGGGTGTCTGATGGCAGCGTTCTTCG | |
| | GATGTTTAGGAGTGGGACTTGGATCTTTGACTATTGG | |
| | AATGCTTGTGGGGATTGTCTCCGCTATCTGTCAGCGT | |
| | AAGCATAAACAAGAAGCTGCACGATGCATTCAGAGA | |
| | GGTATTTTTTACTCCCTTATTTTGGAGCAAATGCAAA | |
| | GATTCCCCAAAGACTTTTTTAGAGATCCTGTGGCGAA | |
| | AAGTATTATGGCCATTCAAGCAGGAGAAGCTTTGGAT | |
| | GAAGGAAAATTGTCTTGGAAAGAAATGCCAAGCATT | |
| | ACAGCTTGCTTAGGAAGAGAGGGATTAGATGCTCAA | |
| | GCCTATTCCTTTATTTCTTCTTCTCCCTTGGATGGTCG | |
| | TATAGAAGAAGCTTTTCGATGA | |
| 18 | MVSFDLSVTTTNIGAGYDDIQRMLNGVTCSSGGMGLLT | TC0412; HP; wild |
| | PSACSPMSSFCSSNQPYSARDLKNRIHQFCQHSGPITGFY | type |
| | SLYNEKIMFEEALLVPTVLEAVESTFWISALSRLGGERPS | |
| | TFDTVILSFFVGLISLVCGAMFVGIVSSAVKIYRLMQTM | |
| | RQARTLNENVQRLLAPQATNMRSAFAKLKGIVASKAL | |
| | DQVEQGYRKERNRMITSFVANALITIAFCALLASVILSAF | |
| | FIGGASGCLMAAFFGCLGVGLGSLTIGMLVGIVSAICQR | |
| | KHKQEAARCIQRGIFYSLILEQMQRFPKDFFRDPVAKSI | |
| | MAIQAGEALDEGKLSWKEMPSITACLGREGLDAQAYSF | |
| | ISSSPLDGRIEEAFR | |
| 19 | ATGTTAAACGGTGTGACTTGTTCTTCCGGAGGAATGG | TC0412; HP; |
| | GATTATTAACTCCTTCCGCATGTTCTCCAATGAGTAG | variant |
| | TTTCTGTTCTTCTAACCAGCCCTATAGTGCTAGAGATT | |
| | TAAAGAATCGCATTCACCAGTTTTGTCAGCATTCGGG | |
| | TCCTATTACGGGATTTTATTCCTTGTACAATGAGAAG | |
| | ATTATGTTTGAGGAGGCTCTTCTTGTTCCGACTGTTCT | |
| | TGAAGCTGTAGAGTCAACTTTTTGGATATCAGCTCTT | |
| | TCTCGTTTAGGAGGAGAGCGTCCTTCGACTTTTGACA | |
| | CGGTAATTCTGAGCTTTTTTGTTGGGCTTATTTCTTTG | |
| | GTGTGCGGGGCGATGTTTGTTGGTATTGTTTCTAGTG | |
| | CCGTGAAAATTTATCGTCTTATGCAGACAATGAGGCA | |
| | AGCACGTACTTTGAATGAAAATGTGCAAAGACTATTA | |
| | GCTCCACAAGCTACGAACATGCGGTCTGCTTTTGCAA | |
| | AACTTAAAGGGATTGTTGCTTCGAAAGCGCTTGATCA | |
| | AGTAGAGCAAGGTTATCGCAAATTCAGAAATCGAAT | |
| | GATTACTTCTTTTGTTGCTAATGCTCTAATTACAATAG | |
| | CCTTTTGTGCTTTATTAGCATCTGTAATTCTTTCTGCT | |
| | TTCTTCATTGGTGGAGCAAGTGGGTGTCTGATGGCAG | |
| | CGTTCTTCGGATGTTTAGGAGTGGGACTTGGATCTTT | |
| | GACTATTGGAATGCTTGTGGGGATTGTCTCCGCTATC | |
| | TGTCAGCGTAAGCATAAACAAGAAGCTGCACGATGC | |
| | ATTCAGAGAGGTATTTTTTACTCCCTTATTTTGGAGCA | |
| | AATGCAAAGATTCCCCAAAGACTTTTTTAGAGATCCT | |
| | GTGGCGAAAAGTATTATGGCCATTCAAGCAGGAGAA | |
| | GCTTTGGATGAAGGAAAATTGTCTTGGAAAGAAATG | |
| | CCAAGCATTACAGCTTGCTTAGGAAGAGAGGGATTA | |
| | GATGCTCAAGCCTATTCCTTTATTTCTTCTTCTCCCTT | |
| | GGATGGTCGTATAGAAGAAGCTTTTCGATGA | |
| 20 | MLNGVTCSSGGMGLLTPSACSPMSSFCSSNQPYSARDL | TC0412; HP; |
| | KNRIHQFCQHSGPITGFYSLYNEKIMFEEALLVPTVLEA | variant (missing |
| | VESTFWISALSRLGGERPSTFDTVILSFFVGLISLVCGAM | the first 21 amino |
| | FVGIVSSAVKIYRLMQTMRQARTLNENVQRLLAPQATN | acids compared to |
| | MRSAFAKLKGIVASKALDQVEQGYRKERNRMITSFVAN | the wildtype) |
| | ALITIAFCALLASVILSAFFIGGASGCLMAAFFGCLGVGL | |
| | GSLTIGMLVGIVSAICQRKHKQEAARCIQRGIFYSLILEQ | |
| | MQRFPKDFFRDPVAKSIMAIQAGEALDEGKLSWKEMPS | |
| | ITACLGREGLDAQAYSFISSSPLDGRIEEAFR | |
| 21 | ATGCGCAGATCTGTTTGTTACGTTACTCCTTCAGTTGC | TC0708; |
| | TAGGGCTGGTCAAATTTCTACCTGGAGGTTTGAATAT | AAS39521; HP, |
| | TCTTCAGCTAATTTCCTTCCCGAGGGGACATTATTAA | metallohydrolase |
| | AATTTGATTTAGGAATAGATGGACGTCCTATAGACTG | domain; wild type |
| | GGAGATTCCTTCTACAGACCTCTCTCAACCATGTAAT | |
| | ACGATCTACTTGGAAACTCCCTCTGAGAGTATCGTAA | |
| | CAGCAAAAGCTGTTTATGCTCCCGGATCCTACATCCC | |
| | TACATTTGAATTTATTCTTCCTTGCGAAATAGAAGCA | |
| | GGAGACACCTTCTCCATCATTCTTGGAAGTTCTCCTA | |
| | ATTTTCCTCAAGAAGATGCTTCTGGCAACGGAGCCCA | |
| | ATTATTTACCCAGCGTCGTAAACCTTTTTCCCTTTACG | |
| | TTGATCCAACGGGAAAAGGAAATTTTGAAGATCCTG | |
| | ATATCTTCACAATAGACATCCGCGGAAATGTTTTAAA | |
| | AAATATCCGTATCTTTGCTCCTTCTTATGTGGTCAAAA | |
| | ATAAGCGCTTTGATATTACAGTTCGCTTCGAAGATGA | |
| | ATTTGGAAATTTAACAAACTTTTCCCCAGAAGAGACC | |
| | CAGATCGAACTTTCGTATGAGCATCTGCGAGAAAATC | |
| | TCAGTTGGCAATTATTTATTCCTGAAACAGGATTCGT | |
| | TATTCTCCCGAATCTTTATTTCAATGAACCTGGGATTT | |
| | ACCGTATTCAATTACGCAATCAAGCCACCAAAGAGA | |
| | TTTTTACGTCTGCTCCAATCAAATGTTTTACAGAAAC | |
| | GTCACCCCATCTTTTATGGGGTCTGCTGCATGGAGAA | |
| | TCTGAGCGTGTAGATTCGGAAGGCAATATTGAATCCT | |
| | GCTTACGTTATTTCCGAGATGACTGCGCTTTGAACTT | |
| | CTTTGCCACATCTTCTTTCGAAATTCAAGATGGGCTT | |
| | ACTCCCGAAACGATTAAAAGCATTAATCAAACGGTCT | |
| | CTGATTTCAATGAAGAAGATCGTTTCATTGCCCTGTC | |
| | GGGGGCACAATATGTTTCTGAAGAACCTGGCGAGGG | |
| | AATTCGTGAAGTCCTTCTAATAAAAGAGCCTAAATCT | |
| | CCAGGAAAGCACAAAGAGTGTAAGTTATTCCCCCTAT | |
| | CTAAACTATATAAGCAATCAACCAGTCATGAGTTAAT | |
| | TTCGATCCCTAGCTTTACTGCATCGAAAAAATTTGGT | |
| | TGTGATTTTCAAAATTTCCATGCCGAATTTGAGCGAG | |
| | TTGTAGAGATTTACAATGCCTGGGGATGTTCTGAAAG | |
| | AACTGAAGCTGAAGGGAATCCATTCCCTATTAAAGGT | |
| | TCTATCGACTCAGAGAATCCAGAAGGGACTATTTTGT | |
| | CTGCTTTAAAGAGAAATCTACGTTTTGGATTTGTAGC | |
| | TGGCGGTCTCGATGATAGAAACTTGTACAGTAACTTT | |
| | TTTGATTCCGATCAACAGCAATACTCTCCTGGATTAA | |
| | CTGCAGTCATTTGTAATAAATATTCTCGTGATTCTCTG | |
| | CTTGAAGCTCTATACCAACGACAATGCTATGCGACAA | |
| | CAGGTCAAAGAATCATTGTAAGTTTTCAAATTACATC | |
| | GGCTCCTATGGGTTCTGAGCTCTCCACAGCTATCAAA | |
| | CCAGGACTAATGATTAATAGACATATTTCAGGATACG | |
| | TAGCGGGGACTGCAAAGATTTCTACTATCGAAATCAT | |
| | CCGTAATGGAGATACACTCCACACCTTCTACCCTGAT | |
| | GGCAACAATTTTGAATATGAGTATGACGATCTGACTC | |
| | CATTTGCTCAGGCTACTCTTGCAGATCCTAAAAATGG | |
| | GGCTCCTTTTGCCTTTTATTACTTACGAGTAACTCAAG | |
| | AAAATGGAGCTATGGCATGGAGTTCTCCTATTTGGAT | |
| | AGATCTCAACTAA | |
| 22 | MRRSVCYVTPSVARAGQISTWRFEYSSANFLPEGTLLK | TC0708; HP, |
| | FDLGIDGRPIDWEIPSTDLSQPCNTIYLETPSESIVTAKAV | metallohydrolase |
| | YAPGSYIPTFEFILPCEIEAGDTFSIILGSSPNFPQEDASGN | domain; wild type |
| | GAQLFTQRRKPFSLYVDPTGKGNFEDPDIFTIDIRGNVL | |
| | KNIRIFAPSYVVKNKRFDITVRFEDEFGNLTNFSPEETQI | |
| | ELSYEHLRENLSWQLFIPETGFVILPNLYFNEPGIYRIQLR | |
| | NQATKEIFTSAPIKCFTETSPHLLWGLLHGESERVDSEG | |
| | NIESCLRYFRDDCALNFFATSSFEIQDGLTPETIKSINQTV | |
| | SDFNEEDRFIALSGAQYVSEEPGEGIREVLLIKEPKSPGK | |
| | HKECKLFPLSKLYKQSTSHELISIPSFTASKKFGCDFQNF | |
| | HAEFERVVEIYNAWGCSERTEAEGNPFPIKGSIDSENPE | |
| | GTILSALKRNLRFGFVAGGLDDRNLYSNFFDSDQQQYS | |
| | PGLTAVICNKYSRDSLLEALYQRQCYATTGQRIIVSFQIT | |
| | SAPMGSELSTAIKPGLMINRHISGYVAGTAKISTIEIIRNG | |
| | DTLHTFYPDGNNFEYEYDDLTPFAQATLADPKNGAPFA | |
| | FYYLRVTQENGAMAWSSPIWIDLN | |
| 23 | ATCGCAGATCTGTTTGTTACGTTACTCCTTCAGTTGCT | TC0708; HP, |
| | AGGGCTGGTCAAATTTCTACCTGGAGGTTTGAATATT | metallohydrolase |
| | CTTCAGCTAATTTCCTTCCCGAGGGGACATTATTAAA | domain; variant |
| | ATTTGATTTAGGAATAGATGGACGTCCTATAGACTGG | (no protein will be |
| | GAGATTCCTTCTACAGACCTCTCTCAACCATGTAATA | produced due to |
| | CGATCTACTTGGAAACTCCCTCTGAGAGTATCGTAAC | the deletion of the |
| | AGCAAAAGCTGTTTATGCTCCCGGATCCTACATCCCT | 3rd G in the Start |
| | ACATTTGAATTTATTCTTCCTTGCGAAATAGAAGCAG | codon since there |
| | GAGACACCTTCTCCATCATTCTTGGAAGTTCTCCTAA | is no alternative |
| | TTTTCCTCAAGAAGATGCTTCTGGCAACGGAGCCCAA | start codon) |
| | TTATTTACCCAGCGTCGTAAACCTTTTTCCCTTTACGT | |
| | TGATCCAACGGGAAAAGGAAATTTTGAAGATCCTGA | |
| | TATCTTCACAATAGACATCCGCGGAAATGTTTTAAAA | |
| | AATATCCGTATCTTTGCTCCTTCTTATGTGGTCAAAAA | |
| | TAAGCGCTTTGATATTACAGTTCGCTTCGAAGATGAA | |
| | TTTGGAAATTTAACAAACTTTTCCCCAGAAGAGACCC | |
| | AGATCGAACTTTCGTATGAGCATCTGCGAGAAAATCT | |
| | CAGTTGGCAATTATTTATTCCTGAAACAGGATTCGTT | |
| | ATTCTCCCGAATCTTTATTTCAATGAACCTGGGATTTA | |
| | CCGTATTCAATTACGCAATCAAGCCACCAAAGAGATT | |
| | TTTACGTCTGCTCCAATCAAATGTTTTACAGAAACGT | |
| | CACCCCATCTTTTATGGGGTCTGCTGCATGGAGAATC | |
| | TGAGCGTGTAGATTCGGAAGGCAATATTGAATCCTGC | |
| | TTACGTTATTTCCGAGATGACTGCGCTTTGAACTTCTT | |
| | TGCCACATCTTCTTTCGAAATTCAAGATGGGCTTACT | |
| | CCCGAAACGATTAAAAGCATTAATCAAACGGTCTCTG | |
| | ATTTCAATGAAGAAGATCGTTTCATTGCCCTGTCGGG | |
| | GGCACAATATGTTTCTGAAGAACCTGGCGAGGGAAT | |
| | TCGTGAAGTCCTTCTAATAAAAGAGCCTAAATCTCCA | |
| | GGAAAGCACAAAGAGTGTAAGTTATTCCCCCTATCTA | |
| | AACTATATAAGCAATCAACCAGTCATGAGTTAATTTC | |
| | GATCCCTAGCTTTACTGCATCGAAAAAATTTGGTTGT | |
| | GATTTTCAAAATTTCCATGCCGAATTTGAGCGAGTTG | |
| | TAGAGATTTACAATGCCTGGGGATGTTCTGAAAGAAC | |
| | TGAAGCTGAAGGGAATCCATTCCCTATTAAAGGTTCT | |
| | ATCGACTCAGAGAATCCAGAAGGGACTATTTTGTCTG | |
| | CTTTAAAGAGAAATCTACGTTTTGGATTTGTAGCTGG | |
| | CGGTCTCGATGATAGAAACTTGTACAGTAACTTTTTT | |
| | GATTCCGATCAACAGCAATACTCTCCTGGATTAACTG | |
| | CAGTCATTTGTAATAAATATTCTCGTGATTCTCTGCTT | |
| | GAAGCTCTATACCAACGACAATGCTATGCGACAACA | |
| | GGTCAAAGAATCATTGTAAGTTTTCAAATTACATCGG | |
| | CTCCTATGGGTTCTGAGCTCTCCACAGCTATCAAACC | |
| | AGGACTAATGATTAATAGACATATTTCAGGATACGTA | |
| | GCGGGGACTGCAAAGATTTCTACTATCGAAATCATCC | |
| | GTAATGGAGATACACTCCACACCTTCTACCCTGATGG | |
| | CAACAATTTTGAATATGAGTATGACGATCTGACTCCA | |
| | TTTGCTCAGGCTACTCTTGCAGATCCTAAAAATGGGG | |
| | CTCCTTTTGCCTTTTATTACTTACGAGTAACTCAAGAA | |
| | AATGGAGCTATGGCATGGAGTTCTCCTATTTGGATAG | |
| | ATCTCAACTAA | |

TABLE 4
Mutations.

| GENE/ | | PREDICTED | AMINO |
|---|---|---|---|
| ACCESSION | NUCLEIC ACID | GENE | ACID |
| No. | MUTATION | FUNCTION | CHANGE |
| TC0168 | C to G at position 38 | Ribosomal | T13R |
| AAF39043.1 | of SEQ ID NO: 1 | protein | |
| | Single nucleotide variant | (rpmH) | |
| TC0341 | T to A at position 1022 | ABC | L341X; |
| AAF73548.1 | of SEQ ID NO: 5 | transporter | resulting |
| | Stop gain | | in premature |
| | | | termination |
| TC0342 | A is deleted at 938 of | ABC | Q313 Hfs*4 |
| AAF39203 | SEQ ID NO: 9 | transporter | |
| | Frame shift deletion | | |
| TC0408 | C to A at position 839 | | P280Q |
| AAF739264; | of SEQ ID NO: 13 | | |
| AAF3926.2 | Single nucleotide variant | | |
| TC0412 | AATGGTAAGTTT | | M1 loss, |
| AAF39268 | deleted at positions 1-11 | | restart at |
| | of SEQ ID NO: 17 | | M23 or M24 |
| | Startloss | | |
| TC0708 | G deletion at position 3 | | Protein not |
| AAS39521 | of SEQ ID NO: 21 | | made |
| | Loss of start codon | | |

### Pharmaceutical Compositions

As disclosed herein, are pharmaceutical compositions, comprising the isolated Chlamydia muridarum cell described here and a pharmaceutical acceptable carrier. In some aspects, the pharmaceutical compositions can comprise any the polypeptides disclosed herein and a pharmaceutical acceptable carrier. In some aspects, the polypeptides can have the amino acid sequence of SEQ ID NOs: 3, 6, 10 and/or 14.

By “pharmaceutically acceptable carrier” is meant a carrier that is compatible with other ingredients in the pharmaceutical composition and that is not harmful or deleterious to the subject. The carrier can be a solid or a liquid, or both, and can be formulated with the composition disclosed herein as a unit-dose formulation, for example, a tablet, which may contain from about 0.01 or 0.5% to about 95% or 99% by weight of the composition. The pharmaceutical compositions can be prepared by any of the well-known techniques of pharmacy including, but not limited to, admixing the components, optionally including one or more accessory ingredients. In some aspects, the pharmaceutically acceptable carrier can be sterile and would be deemed suitable for administration into human subjects according to regulatory guidelines for pharmaceutical compositions comprising the carrier.

In some aspects, the pharmaceutical composition can be formulated for oral administration. The compositions of the present disclosure also contain a therapeutically effective amount of an isolated Chlamydia muridarum cell as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

A “pharmaceutically acceptable” component such as a salt, carrier, excipient or diluent of a composition disclosed herein can be a component that (i) is compatible with the other ingredients of the composition in that it can be combined with the compositions disclosed herein without rendering the composition unsuitable for its intended purpose, and (ii) is suitable for use with subjects as provided herein without undue adverse side effects (such as toxicity, irritation, and allergic response), Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

As disclosed herein, the compositions described herein can be administered to a cell of a subject or to a subject either in vivo or ex vivo. For administration to a cell of the subject in vivo, as well as for administration to the subject, the Chlamydia cells and/or compositions can be administered orally, intranasally, intravaginally, intrarectally, intragastrically, intraurethrally, intraocularly, parenterally (e.g., intravenously), by intramuscular injection, by intraperitoneal injection, subcutaneous injection, transdermally, extracorporeally, topically or the like. Also, in some aspects, the compositions can be pulsed onto dendritic cells, which are isolated or grown from a subject's cells, according to methods well known in the art, or onto bulk peripheral blood mononuclear cells (PBMC) or various cell subfractions thereof from a subject.

The exact amount(s) of the composition(s) described herein that will be required will vary from subject to subject, depending on the species, age, weight and general condition of the subject, the particular composition used, its mode of administration and the like. Thus, it is not possible to specify an exact amount for every composition. However, effective amount can be determined by one of ordinary skill in the art using routine experimentation given the teachings herein and that are well known in the art.

As an example, to a subject diagnosed with Chlamydia infection or known to be at risk of being infected with Chlamydia or in whom it is desirable to induce an immune response to Chlamydia, about 1000 to about 1,000,000 of the Chlamydia cells as disclosed herein can be administered (e.g., intravaginally and/or intranasally for inducing mucosal immunity) and can be in combination with or without an adjuvant, at one to three hour/day/week intervals until an evaluation of the subject's clinical parameters indicate that the subject is not infected by Chlamydia and/or the subject demonstrates the desired immunological response.

In some aspects, to a subject diagnosed with Chlamydia infection or known to be at risk of being infected with Chlamydia or in whom it is desirable to induce an immune response to Chlamydia, about 1000 to about 100,000,000 of the Chlamydia cells as disclosed herein can be administered orally for inducing mucosal immunity, and can be in combination with or without an adjuvant, at one to three hour/day/week intervals until an evaluation of the subject's clinical parameters indicate that the subject is not infected by Chlamydia and/or the subject demonstrates the desired immunological response.

Alternatively, a polypeptide and/or fragment described herein can be pulsed onto dendritic cells at a concentration of between about 10-100 μM and the dendritic cells can be administered to the subject intravenously at the same time intervals. The treatment can be continued or resumed if the subject's clinical parameters indicate that Chlamydia infection is present and/or the desired immunological response is diminished or no longer present and can be maintained until the infection is no longer detected by these parameters and/or until the desired immunological response is achieved or re-established.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the bi-functional allosteric protein-drug molecules. Thus, compositions can be prepared for parenteral administration that includes bi-functional allosteric protein-drug molecules dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Parenteral administration of the peptides, polypeptides, nucleic acids and/or vectors described herein, if used, can be generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained. See, e.g., U.S. Pat. No. 3,610,795, which is incorporated by reference herein in its entirety.

The efficacy of treating or preventing Chlamydia infection by the methods described herein can be determined by detecting a clinical improvement as indicated by a change in the subject's symptoms and/or clinical parameters, as would be well known to one of skill in the art.

The pharmaceutical compositions described herein include those suitable for oral, intranasal, rectal, topical, inhalation (e.g., via an aerosol) buccal (e.g., sub-lingual), vaginal (e.g., vaginal ring), rectal, intraurethral, parenteral (e.g., subcutaneous, intramuscular, intradermal, intraarticular, intrapleural, intraperitoneal, intracerebral, intraarterial, or intravenous), topical (i.e., both skin and mucosal surfaces, including but not limited to vaginal, urethral, rectal, labial, respiratory, oral, nasal, airway surfaces, etc.) and transdermal administration. The compositions herein can also be administered via a skin scarification method or transdermally via a patch, liquid or gel. The compositions can be delivered subdermally in the form of a biodegradable material that releases the compositions over time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition disclosed herein; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets. Such formulations can be prepared by any suitable method of pharmacy, which includes the step of bringing into association the composition and a suitable carrier (which can contain one or more accessory ingredients as described herein). In some aspects, the pharmaceutical composition can be prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets can be prepared by compressing, in a suitable machine, the composition in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Pharmaceutical compositions suitable for buccal (sub-lingual) administration include lozenges comprising the composition disclosed herein in a flavored base, usually sucrose and acacia or tragacanth; and pastilles comprising the composition in an inert base such as gelatin and glycerin or sucrose and acacia.

In some aspects, the pharmaceutical compositions suitable for parenteral administration can comprise sterile aqueous and non-aqueous injection solutions of the composition disclosed herein, which preparations can be isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In some aspects, the compositions disclosed herein can be administered to the mucous membranes of a subject (e.g., via intravaginal administration). The formulations can be prepared in unit dosage form and can be prepared by any of the methods well known in the art. For example, formulations can be administered to the mucosa as a liquid, spray, ointment, gel and/or mist.

The compositions can be presented in unit-dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition disclosed herein. When the composition is substantially water-insoluble, a sufficient amount of emulsifying agent, which is physiologically and pharmaceutically acceptable, can be included in sufficient quantity to emulsify the composition in an aqueous carrier. In some aspects, the emulsifying agent can be phosphatidyl choline.

Pharmaceutical compositions suitable for rectal administration can be provided as unit dose suppositories. These can be prepared by admixing the composition with one or more conventional solid carriers, such as for example, cocoa butter and then shaping the resulting mixture.

In some aspects, pharmaceutical compositions disclosed herein suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some aspects, for example, topical delivery can be performed by mixing a pharmaceutical composition disclosed herein with a lipophilic reagent (e.g., DMSO) that is capable of passing into the skin.

In some aspects, pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition disclosed herein. Suitable formulations can comprise citrate or bis\tris buffer (pH 6) or ethanol/water and can contain from 0.1 to 0.2M active ingredient.

In some aspects, the frequency of administration of a composition disclosed herein can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some aspects, one, two, three or four doses over the lifetime of a subject can be adequate to achieve the desired therapeutic effect. In some aspects, alternate day dosing can be employed (e.g., every other day). The amount and frequency of administration of the composition disclosed herein will vary depending on the particular condition being treated or to be prevented and the desired therapeutic effect.

In some aspects, the compositions disclosed herein can comprise a protein and/or fragment and/or epitope thereof of a different pathogenic organism in any combination (e.g., a pathogenic organism that is sexually transmitted, including but not limited to: Trichomonas (e.g., Trichomonas vaginalis); a pathogenic yeast or fungus (e.g., Candida albicans), Neisseria (e.g., N. gonorrhea), Treponema pallidum, and pathogenic viruses (e.g., herpes simplex virus (HSV), human immunodeficiency virus (HIV), human papilloma virus (HPV)).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of the cells disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to Chlamydia infection or a disease or disorder associated with a Chlamydia infection.

The pharmaceutical compositions described herein can be administered to the subject (e.g., a human patient) in an amount sufficient to delay, reduce, or preferably prevent the onset of clinical disease. Accordingly, in some aspects, the patient can be a human subject or patient. In therapeutic applications, compositions can be administered to a subject (e.g., a human patient) already with or diagnosed with a disease (e.g., Chlamydia infection or a disease or disorder associated with a Chlamydia infection) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the disease is delayed, hindered, or prevented, or the disease or a symptom of the disease is ameliorated. One or more of the symptoms can be less severe. Recovery can be accelerated in an individual who has been treated.

In some aspects, the disease or disorder associated with a Chlamydia genital infection can be pelvic inflammatory disease, tubal factor infertility, ectopic pregnancy, or chronic pelvic pain.

In some aspects, the disease or disorder associated with a Chlamydia ocular infection can be trachoma or conjunctivitis.

In some aspects, the disease or disorder associated with a Chlamydia airway infection can be pneumonia.

Amounts effective for this use can depend on the severity of the disease and the weight and general state and health of the subject. Suitable regimes for initial administration and booster administrations are typified by an initial administration followed by repeated doses at one or more hourly, daily, weekly, or monthly intervals by a subsequent administration. For therapeutic uses, the compositions can include a pharmaceutically acceptable excipient. Such compositions can be formulated without undue experimentation for administration to a mammal, including humans, as appropriate for the particular application. Additionally, proper dosages of the compositions can be determined without undue experimentation using standard dose-response protocols. For example, a subject can receive any of the compound or compositions disclosed herein one or more times per, month (e.g., 2, 3, 4, 5, 6, or 7 or more times per week).

The total effective amount of any of cells in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every day, every 2-4 days, 1-2 weeks, 1-2 a month or 1-2 a year). Alternatively, continuous intravenous infusions sufficient to maintain therapeutically effective concentrations in the blood are also within the scope of the present disclosure.

The therapeutically effective amount of any of the cells or compositions disclosed herein present within the pharmaceutical compositions described herein and used in the methods as disclosed herein applied to mammals (e.g., humans) can be determined by one of ordinary skill in the art with consideration of individual differences in age, weight, and other general conditions (as mentioned herein).

Kits

Disclosed herein a kit comprising the compositions of this invention. It would be well understood by one of ordinary skill in the art that the kit of this invention can comprise one or more containers and/or receptacles to hold the reagents (e.g., cells, antibodies, antigens, nucleic acids) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

In some aspects, the compositions and kits can also include other medicinal agents, pharmaceutical agents, carriers, diluents, immunostimulatory cytokines, etc. Actual methods of preparing such dosage forms are known, or will be apparent, to those skilled in this art.

Methods of Treatment

Disclosed herein are methods of treating and/or preventing a disorder associated with or caused by Chlamydial infection in a subject. Also disclosed herein are methods for ameliorating the pathological conditions associated with chlamydial infection in a subject. In some aspects, the methods can comprise administering to the subject an effective amount of an isolated Chlamydia muridarum cell as described herein or the composition comprising the Chlamydia muridarum cell as described herein.

Disclosed herein are methods of treating and/or prevent infection and/or disease caused by any chlamydial species that can infect a subject, including, for example, but not limited to Chlamydia trachomatis, Chlamydia pneumoniae, Chlamydia muridarum, Chlamydia psittaci, Chlamydophila abortus and/or Chlamydia caviae.

In some aspects, the methods can comprise identifying a patient in need of treatment. In some aspects, the method can comprise administering to the subject a therapeutically effective amount of a pharmaceutical composition (or cell or polypeptide) as disclosed herein.

In some aspects, the subject can have or be suspected of having a lysosomal storage disorder, such as a mucopolysaccharidosis disorder (e.g., Sly syndrome [13-glucuronidase], Hurler Syndrome [a-L-iduronidase], Scheie Syndrome [a-L-iduronidase], Hurler-Scheie Syndrome [a-L-iduronidase], Hunter's Syndrome [iduronate sulfatase], Sanfilippo Syndrome A [heparan sulfamidase], B [N-acetylglucosaminidase], C [acetyl-CoA:a-glucosaminide acetyltransferase], D [N-acetylglucosamine 6-sulfatase], Morquio Syndrome A [galactose-6-sulfate sulfatase], [13-galactosidaset Maroteaux-Lamy Syndrome [N-acetylgalactosamine-4-sulfatase], etc.), Fabry disease (a-galactosidase), Gaucher's disease (glucocerebrosidase), or a glycogen storage disorder (e.g., Pompe disease; lysosomal acid a-glucosidase).

In some aspects, the isolated Chlamydia muridarum cells and compositions disclosed herein can be used in a method described herein to deliver a heterologous nucleic acid to a cell or subject as described herein. For example, the Chlamydia cells disclosed herein can be used to treat a lysosomal storage disorder such as a mucopolysaccharidosis disorder (e.g., Sly syndrome [13-glucuronidase], Hurler Syndrome [a-L-iduronidase], Scheie Syndrome [a-L-iduronidase], Hurler-Scheie Syndrome [a-L-iduronidase], Hunter's Syndrome [iduronate sulfatase], Sanfilippo Syndrome A [heparan sul-famidase], B [N-acetylglucosaminidase], C [acetyl-CoA:a-glucosaminide acetyltransferase], D [N-acetylglucosamine 6-sulfatase], Morquio Syndrome A [galactose-6-sulfate sulfatase], [13-galactosidaset Maroteaux-Lamy Syndrome [N-acetylgalactosamine-4-sulfatase], etc.), Fabry disease (a-galactosidase), Gaucher's disease (glucocerebrosidase), or a glycogen storage disorder (e.g., Pompe disease; lysosomal acid a-glucosidase).

Disclosed herein are methods eliciting an immune response to Chlamydia in a subject. In some aspects, the methods can comprise administering to the subject an effective amount of one or more of the Chlamydia muridarum cells or the compositions disclosed herein. In some aspects, the Chlamydia muridarum cells or the compositions can comprise a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion at M1-R22 or a deletion of M1-P33 in open reading frame TC0412, wherein the amino acid numbering is based on the amino acid sequence of SEQ ID NO: 18; and/or f) a deletion at M1 in open reading frame TC0708, wherein the amino acid numbering is based on the amino acid sequence of SEQ ID NO: 22, wherein the Chlamydia muridarum cell has a phenotype due to the mutation, deletion or substitution of a), b), c), d), e) and/or f) of attenuated pathogenicity. In some aspects, the substitution of a) can be the substitution T13R. In some aspects, the mutation of b) can introduce a stop codon at position 341. In some aspects, the deletion of c) can be a frameshift mutation at Q313. In some aspects, the substitution of d) can be the substitution P280Q. In some aspects, the deletion of e) can be a start-loss mutation. In some aspects, the deletion of f) can be a start-loss mutation.

Disclosed herein are methods of treating or preventing a disorder associated with or caused by a chlamydial infection in a subject. In some aspects, the methods can comprise administering to the subject an effective amount of one or more of the Chlamydia muridarum cells or the compositions disclosed herein. In some aspects, the Chlamydia muridarum cells or the compositions can comprise a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion at M1 in open reading frame TC0412, wherein the amino acid numbering is based on the amino acid sequence of SEQ ID NO: 18; and/or f) a deletion at M1 in open reading frame TC0708, wherein the amino acid numbering is based on the amino acid sequence of SEQ ID NO: 22, wherein the Chlamydia muridarum cell has a phenotype due to the mutation, deletion or substitution of a), b), c), d), e) and/or f) of attenuated pathogenicity. In some aspects, the substitution of a) can be the substitution T13R. In some aspects, the mutation of b) can introduce a stop codon at position 341. In some aspects, the deletion of c) can be a frameshift mutation at Q313. In some aspects, the substitution of d) can be the substitution P280Q. In some aspects, the deletion of e) can be a start-loss mutation. In some aspects, the deletion of f) can be a start-loss mutation.

Disclosed herein are methods of treating, ameliorating and/or preventing a disease or disorder due to Chlamydia infection. In some aspects, the methods can comprise administering to the subject one or more of the polypeptides described herein. In some aspects, the polypeptides can have the amino acid sequence of SEQ ID NOs: 3, 6, 10 and/or 14.

Disclosed herein are methods of reducing the likelihood of infertility due to a chlamydial infection in a subject. In some aspects, the methods can comprise administering to the subject an effective amount of one or more of the Chlamydia muridarum cells or the compositions disclosed herein. In some aspects, the Chlamydia muridarum cells or the compositions can comprise a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion at M1 in open reading frame TC0412, wherein the amino acid numbering is based on the amino acid sequence of SEQ ID NO: 18; and/or f) a deletion at M1 in open reading frame TC0708, wherein the amino acid numbering is based on the amino acid sequence of SEQ ID NO: 22, wherein the Chlamydia muridarum cell has a phenotype due to the mutation, deletion or substitution of a), b), c), d), e) and/or f) of attenuated pathogenicity. In some aspects, the substitution of a) can be the substitution T13R. In some aspects, the mutation of b) can introduce a stop codon at position 341. In some aspects, the deletion of c) can be a frameshift mutation at Q313. In some aspects, the substitution of d) can be the substitution P280Q. In some aspects, the deletion of e) can be a start-loss mutation. In some aspects, the deletion of f) can be a start-loss mutation.

As used herein, “reducing the likelihood of infertility due to Chlamydia infection” is meant that a subject to whom the compositions described herein are administered is prevented from becoming infertile as a result of Chlamydia infection or that the likelihood that the subject will become infertile as a result of being infected by Chlamydia is reduced as compared to the likelihood that an untreated subject will become infertile as a result of being infected by Chlamydia. That infertility is prevented or its likelihood as a result of Chlamydia infection is reduced in a subject can be determined according to protocols described herein and as would be well known in the art.

Disclosed herein are methods of reducing the incidence of hydrosalpinx due to a chlamydial infection in a subject. In some aspects, the methods can comprise administering to the subject an effective amount of one or more of the Chlamydia muridarum cells or the compositions disclosed herein. In some aspects, the Chlamydia muridarum cells or the compositions can comprise a) a substitution at T13 in open reading frame TC0168, wherein the amino acid numbering is based on the amino acid sequence of SEQ ID NO: 3; b) a mutation at L341 in open reading frame TC0341, wherein the amino acid numbering is based on the amino acid sequence of SEQ ID NO: 6; c) a deletion at Q313 in open reading frame TC0342, wherein the amino acid numbering is based on the amino acid sequence of SEQ ID NO: 10; d) a substitution at P280 in open reading frame TC0408, wherein the amino acid numbering is based on the amino acid sequence of SEQ ID NO: 14; e) a deletion at M1 in open reading frame TC0412, wherein the amino acid numbering is based on the amino acid sequence of SEQ ID NO: 18; and/or f) a deletion at M1 in open reading frame TC0708, wherein the amino acid numbering is based on the amino acid sequence of SEQ ID NO: 22, wherein the Chlamydia muridarum cell has a phenotype due to the mutation, deletion or substitution of a), b), c), d), e) and/or f) of attenuated pathogenicity. In some aspects, the substitution of a) can be the substitution T 13R. In some aspects, the mutation of b) can introduce a stop codon at position 341. In some aspects, the deletion of c) can be a frameshift mutation at Q313. In some aspects, the substitution of d) can be the substitution P280Q. In some aspects, the deletion of e) can be a start-loss mutation. In some aspects, the deletion of f) can be a start-loss mutation.

Hydrosalpinx is a result of tubal blockade and subsequent retention of fluid exudate within the tubal lumen. Given that the patency of oviducts is important to allow fertilization of the ovum and sperm, and that the hydrosalpinx fluid is toxic to the ovum, the presence of hydrosalpinx serves as an indirect marker of infertility.

As used herein, “reducing the incidence of hydrosalpinx due to Chlamydia infection” is meant that a subject to whom the compositions described herein are administered will be prevented from or protected against developing hydrosalpinx due to Chlamydia infection or has a reduced likelihood of developing hydrosalpinx due to Chlamydia infection or has a lesser degree of hydrosalpinx due to Chlamydia infection as compared to an untreated subject infected by Chlamydia. That hydrosalpinx due to Chlamydia infection is prevented or its incidence and/or degree are reduced in a subject can be determined according to protocols described herein and as would be well known in the art.

In some aspects, the methods disclosed herein reduces the incidence of hydrosalpinx, oviduct dilatation, and/or cellular infiltration associated with chlamydial infection. Thus, disclosed herein are methods of treating and/or preventing hydrosalpinx, oviduct dilatation, and/or cellular infiltration associated with chlamydial infection in a subject. In some aspects, the methods can comprise administering to the subject a composition and/or cell disclosed herein, with or without an adjuvant.

In some aspects, the chlamydial infection can be Chlamydia trachomatis.

In some aspects, the methods can further comprise administering to the subject an adjuvant. In some aspects, the methods can further comprise administering to the subject an immunostimulatory agent.

Disclosed herein are methods of delivering a heterologous nucleic acid molecule to a subject. In some aspects, the methods can comprise administering to the subject the Chlamydia muridarum cell as described herein. In some aspects, the Chlamydia muridarum cell can comprise a heterologous nucleic acid molecule. In some aspects, the heterologous nucleic acid molecule can encode a therapeutic protein, peptide and/or RNA molecule.

In some aspects, the Chlamydia muridarum cell can be administered to mucosal tissue of the subject.

In some aspects, in any of the methods disclosed herein the Chlamydia muridarum cell can be administered orally to the subject.

In some aspects, in any of the methods disclosed herein, the Chlamydia muridarum cell can be administered to the gastrointestinal (GI) tract of the subject.

Disclosed herein are methods of inducing an immune response to an immunogen in a subject. In some aspects, the methods can comprise administering to the subject the Chlamydia muridarum cell as described herein. In some aspects, the Chlamydia muridarum cell can comprise a heterologous nucleic acid molecule that encodes the immunogen. In some aspects, the immunogen can be a human immunodeficiency virus (HIV) protein or immunogenic fragment thereof. In some aspects, the methods can comprise administering one or more of the polypeptides disclosed herein. In some aspects, the polypeptides can have the amino acid sequence of SEQ ID NOs: 3, 6, 10 and/or 14.

In some aspects, the immune response can include a Th1 immune response. “Th1” refers to a helper T cell response which involves the production of interferon-gamma (IFN-γ), leading to cell-mediated immunity. In some aspects, the immune response can include a Th2 immune response. “Th2” refers to a helper T cell response which involves the release of interleukin 4 (IL-4), leading to humoral immunity. See, e.g., U.S. Patent Application Publication No. 2006/0034871 to Grandi et al., incorporated by reference herein in its entirety for its teachings of Th1 and Th2 responses.

In some aspects, the immune response can includes a gamma interferon (IFN-γ)-dependent protective immune response. Thus, disclosed herein are methods of eliciting a gamma interferon-dependent protective immune response against Chlamydia in a subject. In some aspects, the methods can comprise administering to the subject an effective amount of a composition and/or cell disclosed herein, with or without an adjuvant.

Disclosed herein are methods of treating a gastrointestinal disorder in a subject. In some aspects, the methods can comprise administering to the subject the Chlamydia muridarum cell or composition as described herein. In some aspects, the Chlamydia muridarum cell can comprise a heterologous nucleic acid molecule. In some aspects, the gastrointestinal disorder can be colitis.

In some aspects, “cross-species immunity,” e.g., immunity with respect to multiple species of Chlamydia (e.g., Chlamydia muridarum, Chlamydia trachomatis, etc.) (e.g., cross-species protective immunity) can be accomplished with the methods as described herein. Thus, disclosed herein are methods of eliciting a cross-species immune response in a subject (e.g., to treat and/or prevent chlamydial infection and/or disease) by administering to the subject an effective amount of the chlamydial compositions and/or cells disclosed herein, thereby eliciting a cross-species immune response to a Chlamydia species in the subject.

In some aspects, the compositions disclosed herein can be administered to a subject or to a cell of a subject to impart a therapeutic benefit, such as eliciting an immune response. In some aspects, the methods disclosed herein can comprise administering to the subject and/or to a cell of the subject an effective amount of any composition of the compositions described herein and/or any of the cells described herein. In some aspects, an adjuvant is not administered. In some aspects, an adjuvant can be administered. In some aspects, a cell of the subject can be in vivo or ex vivo. In some aspects, the cell can be, but is not limited to, a CD8+T lymphocyte (e.g., a cytotoxic T lymphocyte), an MHC I-expressing antigen-presenting cell, such as a dendritic cell, a macrophage and/or a monocyte. In some aspects, the cell can also be an antigen presenting cell or other class I MHC-expressing cell which can be contacted with the nucleic acids and/or vectors as described herein under conditions whereby the nucleic acid or vector can be introduced into the cell by standard methods for uptake of nucleic acid and vectors. The nucleic acid encoding the polypeptide and/or fragment disclosed herein can then be expressed and the polypeptide and/or fragment product can be processed within the antigen presenting cell or other MHC I-expressing cell and presented on the cell surface as an MHC I/antigen complex. The antigen presenting cell or other class I MHC-expressing cell can then be contacted with an immune cell of the subject which binds the class I MHC/antigen complex and elicits, for example, an immune response, which treats or prevents Chlamydia infection in the subject.

Detection of an immune response in the subject and/or in the cells of the subject can be carried out according to methods standard in the art for detecting a humoral and/or cellular immune response.

### EXAMPLES

### Example 1: Oral Chlamydia muridarum (Cm) G13 Variant can Induce Protective Immunity Against Both Chlamydia trachomatis (Ct) Infection and Pathogenicity in the Genital Tract

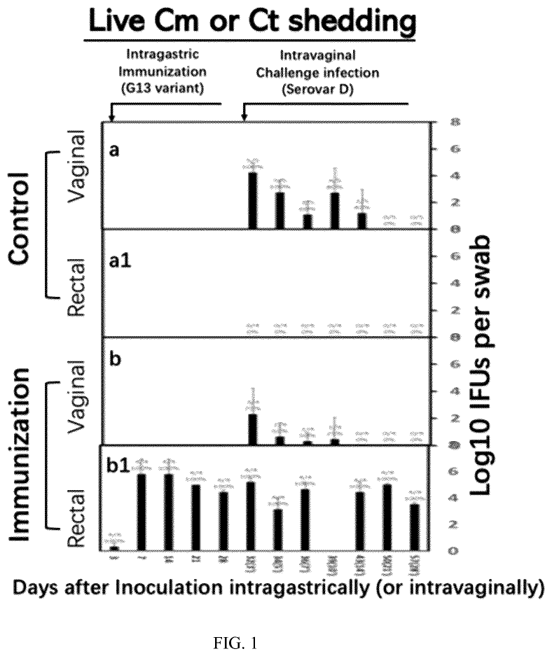

C57BL/6J female mice were each orally inoculated with (FIG. 1, panel b, n=4) or without (FIG. 1, panel a, n=5) 2×10⁵ IFUs of the G13 variant. The G13 variant comprises: Chlamydia muridarum cells comprising: a substitution at T13 in open reading frame TC0168, wherein the amino acid numbering is based on the amino acid sequence of SEQ ID NO: 3; a mutation at L341 in open reading frame TC0341, wherein the amino acid numbering is based on the amino acid sequence of SEQ ID NO: 6; a deletion at Q313 in open reading frame TC0342, wherein the amino acid numbering is based on the amino acid sequence of SEQ ID NO: 10; a substitution at P280 in open reading frame TC0408, wherein the amino acid numbering is based on the amino acid sequence of SEQ ID NO: 14; a deletion of M1-R22 or a deletion of M1-P33 in open reading frame TC0412, wherein the amino acid numbering is based on the amino acid sequence of SEQ ID NO: 18; and a deletion of M1 in open reading frame TC0708, wherein the amino acid numbering is based on the amino acid sequence of SEQ ID NO: 22. Mice were monitored for live chlamydial organisms in both vaginal (FIG. 1, panels a & b) and rectal (FIG. 1, panels a1 & b1) swabs on days 3, 7 and weekly thereafter. The titer of live chlamydial organisms recovered from each swab was expressed as log₁₀ IFUs per swab shown along the Y-axis of FIG. 1. Note that the immunized mice continued to shed live Cm G13 variant organisms following the single oral inoculation but did not allow gut organisms to spread to their genital tract.

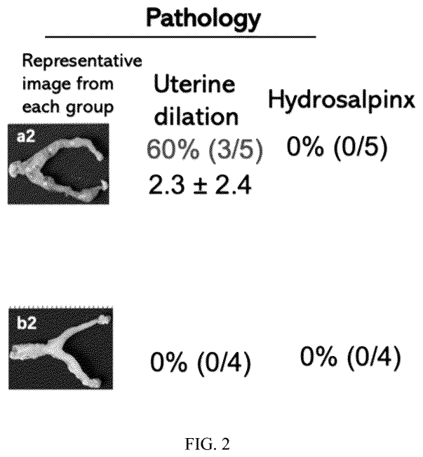

On day 29, the mice were intravaginally challenged with 2×10⁸ IFUs of Ct serovar D. The mice were monitored for live chlamydial organisms from both vaginal and rectal swabs on days 3, 5, 7, 10, 14, 21 and 28 after intravaginal infection as indicated in parenthesis along X-axis of FIG. 1. The titer of live chlamydial organisms recovered from each swab was expressed as log₁₀ IFUs per swab shown along the Y-axis of FIG. 1. Note that the immunized mice significantly reduced live chlamydial organisms in the genital tract with both decreased titers as early as day 3 after challenge infection and shortened genital infection course. On day 28 after intravaginal inoculation, the mice were sacrificed for observing pathology. A representative gross pathology image is shown in FIG. 2 from control (FIG. 2, panel a2) and immunization (FIG. 2, panel b2) group. Sixty percent of control mice developed uterine dilation with a mean score of 2.3. However, none of the control animals developed hydrosalpinx. Most importantly, none of the immunized mice developed any uterine visible uterine pathology.

Thus, oral Cm G13 variant can induce protective immunity against both Ct infection and pathogenicity in the genital tract.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An isolated Chlamydia muridarum cell comprising:
a) a substitution at T13 in open reading frame TC0168, wherein the amino acid numbering is based on the amino acid sequence of SEQ ID NO: 3;
b) a mutation at L341 in open reading frame TC0341, wherein the amino acid numbering is based on the amino acid sequence of SEQ ID NO: 6;
c) a deletion at Q313 in open reading frame TC0342, wherein the amino acid numbering is based on the amino acid sequence of SEQ ID NO: 10;
d) a substitution at P280 in open reading frame TC0408, wherein the amino acid numbering is based on the amino acid sequence of SEQ ID NO: 14;
e) a deletion of M1-R22 or a deletion of M1-P33 in open reading frame TC0412, wherein the amino acid numbering is based on the amino acid sequence of SEQ ID NO: 18; and/or
f) a deletion of M1 in open reading frame TC0708, wherein the amino acid numbering is based on the amino acid sequence of SEQ ID NO: 22,
wherein the Chlamydia muridarum cell has a phenotype due to the mutation, deletion or substitution of a), b), c), d), e) and/or f) of attenuated pathogenicity.

2. The Chlamydia muridarum cell of claim 1, wherein the substitution of a) is the substitution T13R.

3. The Chlamydia muridarum cell of claim 1, wherein the mutation of b) introduces a stop codon at position 341.

4. The Chlamydia muridarum cell of claim 1, wherein the deletion of c) is a frameshift mutation at Q313.

5. The Chlamydia muridarum cell of claim 1, wherein the substitution of d) is the substitution P280Q.

6. The Chlamydia muridarum cell of claim 1, wherein deletion of e) is a start-loss mutation.

7. The Chlamydia muridarum cell of claim 1, wherein deletion of f) is a start-loss mutation.

8. The Chlamydia muridarum cell of any of claims 1-7, further comprising a heterologous nucleic acid molecule.

9. A composition comprising the Chlamydia muridarum cell of any of claims 1-8 and a pharmaceutically acceptable carrier.

10. A method of eliciting an immune response to Chlamydia in a subject, the method comprising administering to the subject an effective amount of one or more of the Chlamydia muridarum cells of claims 1-8 or the composition of claim 9.

11. A method of treating or preventing a disorder associated with or caused by a chlamydial infection in a subject, the method comprising administering to the subject an effective amount of one or more of the Chlamydia muridarum cell of claims 1-8 or the composition of claim 9.

12. A method of reducing the likelihood of infertility due to a chlamydial infection in a subject, the method comprising administering to the subject an effective amount of one or more of the Chlamydia muridarum cell of claims 1-8 or the composition of claim 9.

13. A method of reducing the incidence of hydrosalpinx due to a chlamydial infection in a subject, the method comprising administering to the subject an effective amount of one or more of the Chlamydia muridarum cell of claims 1-8 or the composition of claim 9.

14. The method of any one of claims 11-13, wherein the chlamydial infection is Chlamydia trachomatis.

15. The method of any one of claims 11-13, further comprising administering to the subject an adjuvant.

16. A method of delivering a heterologous nucleic acid molecule to a subject, the method comprising administering to the subject the Chlamydia muridarum cell of claim 8.

17. The method of claim 16, wherein the heterologous nucleic acid molecule encodes a therapeutic protein or therapeutic RNA.

18. The method of claim 16, wherein the Chlamydia muridarum cell is administered to mucosal tissue of the subject.

19. The method of claim 16, wherein the Chlamydia muridarum cell is administered orally to the subject.

20. The method of claim 16, wherein the Chlamydia muridarum cell is administered to the gastrointestinal (GI) tract of the subject.

21. A method of inducing an immune response to an immunogen in a subject, the method comprising administering to the subject the Chlamydia muridarum cell of claim 8, wherein the heterologous nucleic acid molecule encodes the immunogen.

22. The method of claim 21, wherein the immunogen is a human immunodeficiency virus (HIV) protein or immunogenic fragment thereof.

23. A method of treating a gastrointestinal disorder in a subject, the method comprising administering to the gastrointestinal tract of the subject the Chlamydia muridarum cell of claim 8.

24. A polypeptide comprising a substitution at one or more of a) T13 in open reading frame TC0168, wherein the amino acid numbering is based on the amino acid sequence of SEQ ID NO: 3; and/or b) P280 in open reading frame TC0408, wherein the amino acid numbering is based on the amino acid sequence of SEQ ID NO: 14.

25. A polypeptide comprising a deletion at Q313 in open reading frame TC0342, wherein the amino acid numbering is based on the amino acid sequence of SEQ ID NO: 10.

26. A polypeptide comprising a mutation at L341 in open reading frame TC0341, wherein the amino acid numbering is based on the amino acid sequence of SEQ ID NO: 6.

27. A polypeptide comprising a deletion at one or more of a) Q313 in open reading frame TC0342, wherein the amino acid numbering is based on the amino acid sequence of SEQ ID NO: 10; b) M1-R22 or M1-P33 in open reading frame TC0412, wherein the amino acid numbering is based on the amino acid sequence of SEQ ID NO: 20; and/or c) M1 in open reading frame TC0708, wherein the amino acid numbering is based on the amino acid sequence of SEQ ID NO: 22.

28. A composition comprising the polypeptide of any of claims 24-27 in a pharmaceutically acceptable carrier.

29. A method of treating, ameliorating and/or preventing a disease or disorder associated with a Chlamydia infection, the method comprising administering to the subject the isolated polypeptide of any of claims 24-27.

30. The method of claim 29, wherein the polypeptide is administered orally.


SEQUENCE LISTING (23 sequences)

SEQ ID NO: 1 - DNA, 138 bp, synthetic construct
  source 1..138: mol_type = other DNA
gtgaaacgcacctatcaacctagtaaaagaaaacgtacaaattctgtgggatttagagctcgcatggctacaaaaagtggaagaaatcttttaaatcgtcgtcgccgtcacggcagacattccttaattgatctctaa

SEQ ID NO: 2 - DNA, 138 bp, synthetic construct
  source 1..138: mol_type = other DNA
gtgaaacgcacctatcaacctagtaaaagaaaacgtagaaattctgtgggatttagagctcgcatggctacaaaaagtggaagaaatcttttaaatcgtcgtcgccgtcacggcagacattccttaattgatctctaa

SEQ ID NO: 3 - protein, 45 aa, synthetic construct
MKRTYQPSKRKRTNSVGFRARMATKSGRNLLNRRRRHGRHSLIDL

SEQ ID NO: 4 - protein, 45 aa, synthetic construct
MKRTYQPSKRKRRNSVGFRARMATKSGRNLLNRRRRHGRHSLIDL

SEQ ID NO: 5 - DNA, 1344 bp, synthetic construct
  source 1..1344: mol_type = other DNA
atgcttagttgtatattcaaagacacgatcttcctgtccagttttttggccgtttcactgatttgcatgacaacagctttatggggaaccattcttttagtagaaagacgtcccttgttaagcgaaagtctttcgcatgcttgttacccaggtcttttgataggggcccttctttcctacaaagtccctttattttcagattctttgtggatcattatcttgtgcggctgcgcggcttctattttgggatgcttatgcatagcttttttagaaaaaaaacttgctatgcataaagattcagctttgtgtttaattctcgtttcattttttggggtgggggttattcttgttagctatgtcaaagatagctgtcctcttttatataacaagatcaatgcttacttgtatggacaagcggcaactttaggctatgccgaagctaggctggcactcatcattttttgtttatcagcattcgtgctgtggtggtggtatcgacaaatttctgtagcaattttcgatagagaatttgcttattcctgcgggttaagaactcgtacggcggagatgattgttcttgtattcatttcattagtcatagtaagtggtgttcgttctgtagggattttgcttatttcggcaatgtttgttgccccacctttatctgccagacaactctcagataaattaagttcaatactcattttatctagtatttttgggggaatctgtggggctttaggatgttatttttccgtagcgtttacctgccaatctattattgaagggaaggcaacggtaattgtcctcccaactggtccactagtagtattttttgcgggagttcttgttttcttgtgcttaattttctcatggaagactggttggatcacgcgatatatccgtaggaaattatttttattttctcgtgatgaggagcatttattgaagatcttttggtatttgcaagaacaaaatatctatcgtgttagtatgtgggactttgttcgttctagaaagttacaggaatattttgggtccaaagcattttcaggatttaggatgttttggctatgtaagaagggattagtatcttgtttcgatcatcaatgmtttttgacggataaaggagaagccaaagcggctaaattagttcgtgctcatagattatgggagtcttatctggtcagtgaattagattttaacaaaagtaaagtacataatttcgcagaagagatggaacatgttttgactgatgaattggatactactctatcgaagatgctgcaaaatcctgattatgatccacataagcgagagattccaaaactaaaagggaagtaa

SEQ ID NO: 6 - protein, 446 aa, synthetic construct
MLSCIFKDTIFLSSFLAVSLICMTTALWGTILLVERRPLLSESLSHACYPGLLIGALLSKVPLFSDSLWIIILCGCAASILGCLCIAFLEKKLAMHKDSALCLILVSFFGVGVILVSYVKDSCPLLYNKINAYLYGQAATLGYAEARLALIIFCLSAFVLWWWYRQISVAIFDREFAYSCGLRTRTAEMIVLVFISLVIVSGVRSVGILLISAMFVAPPLSARQLSDKLSSILILSSIFGGICGALGCYFSVAFTCQSIIEGKATVIVLPTGPLVVFFAGVLVFLCLIFSWKTGWITRYIRRKLFLFSRDEEHLLKIFWYLQEQNIYRVSMWDFVRSRKLQEYFGSKAFSGFRMFWLCKKGLVSCFDHQXFLTDKGEAKAAKLVRAHRLWESYLVSELDFNKSKVHNFAEEMEHVLTDELDTTLSKMLQNPDYDPHKREIPKLKGK

SEQ ID NO: 7 - DNA, 1344 bp, synthetic construct
  source 1..1344: mol_type = other DNA
atgcttagttgtatattcaaagacacgatcttcctgtccagttttttggccgtttcactgatttgcatgacaacagctttatggggaaccattcttttagtagaaagacgtcccttgttaagcgaaagtctttcgcatgcttgttacccaggtcttttgataggggcccttctttcctacaaagtccctttattttcagattctttgtggatcattatcttgtgcggctgcgcggcttctattttgggatgcttatgcatagcttttttagaaaaaaaacttgctatgcataaagattcagctttgtgtttaattctcgtttcattttttggggtgggggttattcttgttagctatgtcaaagatagctgtcctcttttatataacaagatcaatgcttacttgtatggacaagcggcaactttaggctatgccgaagctaggctggcactcatcattttttgtttatcagcattcgtgctgtggtggtggtatcgacaaatttctgtagcaattttcgatagagaatttgcttattcctgcgggttaagaactcgtacggcggagatgattgttcttgtattcatttcattagtcatagtaagtggtgttcgttctgtagggattttgcttatttcggcaatgtttgttgccccacctttatctgccagacaactctcagataaattaagttcaatactcattttatctagtatttttgggggaatctgtggggctttaggatgttatttttccgtagcgtttacctgccaatctattattgaagggaaggcaacggtaattgtcctcccaactggtccactagtagtattttttgcgggagttcttgttttcttgtgcttaattttctcatggaagactggttggatcacgcgatatatccgtaggaaattatttttattttctcgtgatgaggagcatttattgaagatcttttggtatttgcaagaacaaaatatctatcgtgttagtatgtgggactttgttcgttctagaaagtaacaggaatattttgggtccaaagcattttcaggatttaggatgttttggctatgtaagaagggattagtatcttgtttcgatcatcaatgmtttttgacggataaaggagaagccaaagcggctaaattagttcgtgctcatagattatgggagtcttatctggtcagtgaattagattttaacaaaagtaaagtacataatttcgcagaagagatggaacatgttttgactgatgaattggatactactctatcgaagatgctgcaaaatcctgattatgatccacataagcgagagattccaaaactaaaagggaagtaa

SEQ ID NO: 8 - protein, 340 aa, synthetic construct
MLSCIFKDTIFLSSFLAVSLICMTTALWGTILLVERRPLLSESLSHACYPGLLIGALLSYKVPLFSDSLWIIILCGCAASILGCLCIAFLEKKLAMHKDSALCLILVSFFGVGVILVSYVKDSCPLLYNKINAYLYGQAATLGYAEARLALIIFCLSAFVLWWWYRQISVAIFDREFAYSCGLRTRTAEMIVLVFISLVIVSGVRSVGILLISAMFVAPPLSARQLSDKLSSILILSSIFGGICGALGCYFSVAFTCQSIIEGKATVIVLPTGPLVVFFAGVLVFLCLIFSWKTGWITRYIRRKLFLFSRDEEHLLKIFWYLQEQNIYRVSMWDFVRSRK

SEQ ID NO: 9 - DNA, 951 bp, synthetic construct
  source 1..951: mol_type = other DNA
atgtttgctagtatctctccgtactacggggtatcattttttgagttttttatcgtctttttttcgcgtcttttttctgggaaattgttttatgatcacctgtatatcgatgatattcaagtgattgtattctttgcgatagccgtttcttgttctataatcggaacctttttagttcttaaaaaaatggctatgtatgcaaacgtagtttcccataccatattatttggtttggtatgcgcctgtttgtttactcatcagcttacccatttgtcgatgcaaaatttgacaatagctgccatttcaacaactttattaaccggagcctctatccattttattcgcaatgtatttaaagtagcggaagaggctagtactgctttagttttttctcttttgttttcagccagcttattgcttcttgtgtttttaacaaggaatgctcatgtcgggactgagttagttattggaaatgctgatgcattagctaaaacagatattttcccagtatttttggttttattgamtaatttgggcgtttcttactgcttcttctcgagttttatttgtgtatcttttgataccgtattcgcgttttctttaggtattcgagtcaaattaattgattatcttatgatgtttttgctttctgcttctattgttggagctttcaaagctgtgggcgtgttaatgtcattagcttttctactagtcccaggattaatcgctaagttgatcgcttcctctgttcaggagatgatgggatattccatgatttttggagtgctttcagctttgattgctcctgctctctctagatcgattttatctgttcatgggattggcttatcgacttcagggttagctgtatgtctgttacttgttttttacatagggacgcttgcaactgtttttgtcagaaggcatattttacttcaatcgaaaaattga

SEQ ID NO: 10 - protein, 316 aa, synthetic construct
MFASISPYYGVSFFEFFIVFFSRLFSGKLFYDHLYIDDIQVIVFFAIAVSCSIIGTFLVLKKMAMYANVVSHTILFGLVCACLFTHQLTHLSMQNLTIAAISTTLLTGASIHFIRNVFKVAEEASTALVFSLLFSASLLLLVFLTRNAHVGTELVIGNADALAKTDIFPVFLVLLXNLGVSYCFFSSFICVSFDTVFAFSLGIRVKLIDYLMMFLLSASIVGAFKAVGVLMSLAFLLVPGLIAKLIASSVQEMMGYSMIFGVLSALIAPALSRSILSVHGIGLSTSGLAVCLLLVFYIGTLATVFVRRHILLQSKN

SEQ ID NO: 11 - DNA, 950 bp, synthetic construct
  source 1..950: mol_type = other DNA
atgtttgctagtatctctccgtactacggggtatcattttttgagttttttatcgtctttttttcgcgtcttttttctgggaaattgttttatgatcacctgtatatcgatgatattcaagtgattgtattctttgcgatagccgtttcttgttctataatcggaacctttttagttcttaaaaaaatggctatgtatgcaaacgtagtttcccataccatattatttggtttggtatgcgcctgtttgtttactcatcagcttacccatttgtcgatgcaaaatttgacaatagctgccatttcaacaactttattaaccggagcctctatccattttattcgcaatgtatttaaagtagcggaagaggctagtactgctttagttttttctcttttgttttcagccagcttattgcttcttgtgtttttaacaaggaatgctcatgtcgggactgagttagttattggaaatgctgatgcattagctaaaacagatattttcccagtatttttggttttattgamtaatttgggcgtttcttactgcttcttctcgagttttatttgtgtatcttttgataccgtattcgcgttttctttaggtattcgagtcaaattaattgattatcttatgatgtttttgctttctgcttctattgttggagctttcaaagctgtgggcgtgttaatgtcattagcttttctactagtcccaggattaatcgctaagttgatcgcttcctctgttcaggagatgatgggatattccatgatttttggagtgctttcagctttgattgctcctgctctctctagatcgattttatctgttcatgggattggcttatcgacttcagggttagctgtatgtctgttacttgttttttacatagggacgcttgcaactgtttttgtcagaaggcatattttacttcatcgaaaaattga

SEQ ID NO: 12 - protein, 316 aa, synthetic construct
MFASISPYYGVSFFEFFIVFFSRLFSGKLFYDHLYIDDIQVIVFFAIAVSCSIIGTFLVLKKMAMYANVVSHTILFGLVCACLFTHQLTHLSMQNLTIAAISTTLLTGASIHFIRNVFKVAEEASTALVFSLLFSASLLLLVFLTRNAHVGTELVIGNADALAKTDIFPVFLVLLXNLGVSYCFFSSFICVSFDTVFAFSLGIRVKLIDYLMMFLLSASIVGAFKAVGVLMSLAFLLVPGLIAKLIASSVQEMMGYSMIFGVLSALIAPALSRSILSVHGIGLSTSGLAVCLLLVFYIGTLATVFVRRHILLHRKI

SEQ ID NO: 13 - DNA, 3417 bp, synthetic construct
  source 1..3417: mol_type = other DNA
atgaagcgttctttgtggtacaaaagatttagctattatctccttgtaggattaccgttagctttttttgtcctacttcctaaatttctttccagtgaatccgggaaatatctctttctttctgtactgaataaagaaacggggttgcaatgcgaaattgaacagttacatctatcctggtttggctcccaaacagctaaaaaagtgcgtattcgtggaattgactctgaatccgagattttctctgcagaacaaattattgtaagtggatctcttcctcgtttacttctttacagattccccaaatccctaacgctgactggatggactttacaaatcgatgaatctttatcgataaattctccttccttatatcacctagatcctgggtccctcctttctaaaatagaacgcagcgatattacatccgagttaggatccataaccatgaaaacaatcaaaggatctacgctttctgtttcaggattttatgtaaaaaaaacagcccaacaattactcatccgagctcttacaaaagaaaatgatatacagggttctgtatctgtagaaggagctctttctcctcattttcttcttaatgttcagctttcttctgttcccgcttcactttttaagctctttattgcctccccttctatagatagaatcctatcaacagaggatcttattaacctaacagccaaggctcaccaagaaaaggactctactcttattaccttgaccacagaaggaaatcaaataactgcaaaacttcgaggtcatatacgtgataatacatttttaatcactcaaggaggagcctcatccatagttttgccaccagcaataacctcaagtatcctctctgagttattagcagtagagacctccattcgctcaaaagaagctcacctatttatatctgacgcgaaacttcccctttctatctcaaaatggaattcttctgaattttcattgcaggctaacttgcctcaaatttctgtagaaacatccgatcctaatctctctatccaaacagaaaatacaaaaatttcggtaaaaaaatctgaccgttttacactcattcgatcttcttctgcagcaagactaggaggagcctccccttcttatattcatggatctctttctatagataaccgtaaacactctgcagaatttcatatccaacaatctctcttaccacatacgtatttacgagcgcttcttcctaaacctttcgaaataaatatcccccttgaagtcccttattacactttggatgtcaaagggaaatatcgtaatacccaactctcatgcgaagcttttttagataaccccttattacgcttatcatgcaccctttccggacctttacaaactttgcaatttaatggagaaggagcctacacgttgtctgaacgctggaaagaccgtttctctccttattttctgcaaatgcaaacatcattttctgggaaaatgcattttactcaacgacatatctttttcccaaaactatctgtcaaactctttgcaggagaaaacgaagttttcatccatgggaaatttggtaaagctaatgaagaaattaagccctccaatacttctcttctagtgtatgggaaccttttttctcttcctatggacctctgttcctctaaattcgctccgctgcaaatgacacattcaagcttttccttacattctgatggagggaaaggtgtgcttaaagggaacttaaaattatccataaccgatcctgaattccctcacttatcggaaacaaagctcttaattcccgacattgtgatctcatctttagatcctacatccccttgggatgcggagaatatctccattcaagcatctggagagcttttacaattgcctgtagataggctcattcgattgcagcataaagatgcctccttatcccgttacattggggaaacaggaaaagcctctttccaacttctttactctccagataaagaggaaactatctccatcgcttcaacatttaaaacggatgctttgactggggatttccgctttgttatggatagctcgttatctctgacagagaacacttatggctcactacaatgggaagtttctccggagcgttatagctctttctttgaaaaagcttccagctccccatcctgccttctacatcgccccactaccttccgcctagctctttctaaaatttcctgctcagacaaaaaaacgggatactcttgtctttctctcttgtcagaaggtggaatagaaggaaaactatcctccacgcctttagttttttatgaccatttatccaaagaaaacttcatcgtaaacaatataaatggatctgttcacgcacaaaatctaaatgacctaatccaatataaattcactggaaactgtttagctcctaaccaagataataaatcccctgtatcttttgtcattgaaggtgaagccaaaaatattttaatgaatgaaacaagaagtttaacccaaacagcaacctggacaaacattccgacatcctttatcacgggcattgttcccatttctcctggagttagagctaaactcacctccttagcaggacctaaaatccatatctctatctacaatgatttttataaaggagaaggaccattaaccattaaggtagattctgaaaatctgacaggttatttacctctagagcttacagagcatgctattctgctaaaagaagaccttacagcatctttgcacatcaatgaagaaattaataaggcattccttagagaattcaacccattgatttcagaggggggtgcttattcaaaacatccagtaagtttacgagtaaatccgcaaaatttttatctgcctattaaaccttactctttcgaaaattttcgtattcaatctgcttccctagatttcgggaaaatagagatagagaatacaggaactatgcaagacctctttcaattcttagatgtcgaggcggagcagcaaagagtggaatcttggtttacacctatctttttctctgtacaaaacggtcaaattatctataagcgctttgatgctttaatcgacggacgcatacgcttagctctatggggaaaaaccgacgttatccgagacagattagcaatgactctagggattgatccagaactcattaaaaaactgttccgcaacacgatgctcaaaacgaaaaattttttcttaatcaagattcgcggccctatctcgtcaccagaaattgactggtcgtccgcttacgctagaatcgctctactgaaaagctacacgatcgcgggtcctctgaactcattagcagacaaattattttcgtcattaggagaacccactccttcacagacagtgtccccccttccatgggaagtttctgagtcagagtaa

SEQ ID NO: 14 - protein, 1138 aa, synthetic construct
MKRSLWYKRFSYYLLVGLPLAFFVLLPKFLSSESGKYLFLSVLNKETGLQCEIEQLHLSWFGSQTAKKVRIRGIDSESEIFSAEQIIVSGSLPRLLLYRFPKSLTLTGWTLQIDESLSINSPSLYHLDPGSLLSKIERSDITSELGSITMKTIKGSTLSVSGFYVKKTAQQLLIRALTKENDIQGSVSVEGALSPHFLLNVQLSSVPASLFKLFIASPSIDRILSTEDLINLTAKAHQEKDSTLITLTTEGNQITAKLRGHIRDNTFLITQGGASSIVLPPAITSSILSELLAVETSIRSKEAHLFISDAKLPLSISKWNSSEFSLQANLPQISVETSDPNLSIQTENTKISVKKSDRFTLIRSSSAARLGGASPSYIHGSLSIDNRKHSAEFHIQQSLLPHTYLRALLPKPFEINIPLEVPYYTLDVKGKYRNTQLSCEAFLDNPLLRLSCTLSGPLQTLQFNGEGAYTLSERWKDRFSPYFLQMQTSFSGKMHFTQRHIFFPKLSVKLFAGENEVFIHGKFGKANEEIKPSNTSLLVYGNLFSLPMDLCSSKFAPLQMTHSSFSLHSDGGKGVLKGNLKLSITDPEFPHLSETKLLIPDIVISSLDPTSPWDAENISIQASGELLQLPVDRLIRLQHKDASLSRYIGETGKASFQLLYSPDKEETISIASTFKTDALTGDFRFVMDSSLSLTENTYGSLQWEVSPERYSSFFEKASSSPSCLLHRPTTFRLALSKISCSDKKTGYSCLSLLSEGGIEGKLSSTPLVFYDHLSKENFIVNNINGSVHAQNLNDLIQYKFTGNCLAPNQDNKSPVSFVIEGEAKNILMNETRSLTQTATWTNIPTSFITGIVPISPGVRAKLTSLAGPKIHISIYNDFYKGEGPLTIKVDSENLTGYLPLELTEHAILLKEDLTASLHINEEINKAFLREFNPLISEGGAYSKHPVSLRVNPQNFYLPIKPYSFENFRIQSASLDFGKIEIENTGTMQDLFQFLDVEAEQQRVESWFTPIFFSVQNGQIIYKRFDALIDGRIRLALWGKTDVIRDRLAMTLGIDPELIKKLFRNTMLKTKNFFLIKIRGPISSPEIDWSSAYARIALLKSYTIAGPLNSLADKLFSSLGEPTPSQTVSPLPWEVSESE

SEQ ID NO: 15 - DNA, 3417 bp, synthetic construct
  source 1..3417: mol_type = other DNA
atgaagcgttctttgtggtacaaaagatttagctattatctccttgtaggattaccgttagctttttttgtcctacttcctaaatttctttccagtgaatccgggaaatatctctttctttctgtactgaataaagaaacggggttgcaatgcgaaattgaacagttacatctatcctggtttggctcccaaacagctaaaaaagtgcgtattcgtggaattgactctgaatccgagattttctctgcagaacaaattattgtaagtggatctcttcctcgtttacttctttacagattccccaaatccctaacgctgactggatggactttacaaatcgatgaatctttatcgataaattctccttccttatatcacctagatcctgggtccctcctttctaaaatagaacgcagcgatattacatccgagttaggatccataaccatgaaaacaatcaaaggatctacgctttctgtttcaggattttatgtaaaaaaaacagcccaacaattactcatccgagctcttacaaaagaaaatgatatacagggttctgtatctgtagaaggagctctttctcctcattttcttcttaatgttcagctttcttctgttcccgcttcactttttaagctctttattgcctccccttctatagatagaatcctatcaacagaggatcttattaacctaacagccaaggctcaccaagaaaaggactctactcttattaccttgaccacagaaggaaatcaaataactgcaaaacttcgaggtcatatacgtgataatacatttttaatcactcaaggaggagcctcatccatagttttgcaaccagcaataacctcaagtatcctctctgagttattagcagtagagacctccattcgctcaaaagaagctcacctatttatatctgacgcgaaacttcccctttctatctcaaaatggaattcttctgaattttcattgcaggctaacttgcctcaaatttctgtagaaacatccgatcctaatctctctatccaaacagaaaatacaaaaatttcggtaaaaaaatctgaccgttttacactcattcgatcttcttctgcagcaagactaggaggagcctccccttcttatattcatggatctctttctatagataaccgtaaacactctgcagaatttcatatccaacaatctctcttaccacatacgtatttacgagcgcttcttcctaaacctttcgaaataaatatcccccttgaagtcccttattacactttggatgtcaaagggaaatatcgtaatacccaactctcatgcgaagcttttttagataaccccttattacgcttatcatgcaccctttccggacctttacaaactttgcaatttaatggagaaggagcctacacgttgtctgaacgctggaaagaccgtttctctccttattttctgcaaatgcaaacatcattttctgggaaaatgcattttactcaacgacatatctttttcccaaaactatctgtcaaactctttgcaggagaaaacgaagttttcatccatgggaaatttggtaaagctaatgaagaaattaagccctccaatacttctcttctagtgtatgggaaccttttttctcttcctatggacctctgttcctctaaattcgctccgctgcaaatgacacattcaagcttttccttacattctgatggagggaaaggtgtgcttaaagggaacttaaaattatccataaccgatcctgaattccctcacttatcggaaacaaagctcttaattcccgacattgtgatctcatctttagatcctacatccccttgggatgcggagaatatctccattcaagcatctggagagcttttacaattgcctgtagataggctcattcgattgcagcataaagatgcctccttatcccgttacattggggaaacaggaaaagcctctttccaacttctttactctccagataaagaggaaactatctccatcgcttcaacatttaaaacggatgctttgactggggatttccgctttgttatggatagctcgttatctctgacagagaacacttatggctcactacaatgggaagtttctccggagcgttatagctctttctttgaaaaagcttccagctccccatcctgccttctacatcgccccactaccttccgcctagctctttctaaaatttcctgctcagacaaaaaaacgggatactcttgtctttctctcttgtcagaaggtggaatagaaggaaaactatcctccacgcctttagttttttatgaccatttatccaaagaaaacttcatcgtaaacaatataaatggatctgttcacgcacaaaatctaaatgacctaatccaatataaattcactggaaactgtttagctcctaaccaagataataaatcccctgtatcttttgtcattgaaggtgaagccaaaaatattttaatgaatgaaacaagaagtttaacccaaacagcaacctggacaaacattccgacatcctttatcacgggcattgttcccatttctcctggagttagagctaaactcacctccttagcaggacctaaaatccatatctctatctacaatgatttttataaaggagaaggaccattaaccattaaggtagattctgaaaatctgacaggttatttacctctagagcttacagagcatgctattctgctaaaagaagaccttacagcatctttgcacatcaatgaagaaattaataaggcattccttagagaattcaacccattgatttcagaggggggtgcttattcaaaacatccagtaagtttacgagtaaatccgcaaaatttttatctgcctattaaaccttactctttcgaaaattttcgtattcaatctgcttccctagatttcgggaaaatagagatagagaatacaggaactatgcaagacctctttcaattcttagatgtcgaggcggagcagcaaagagtggaatcttggtttacacctatctttttctctgtacaaaacggtcaaattatctataagcgctttgatgctttaatcgacggacgcatacgcttagctctatggggaaaaaccgacgttatccgagacagattagcaatgactctagggattgatccagaactcattaaaaaactgttccgcaacacgatgctcaaaacgaaaaattttttcttaatcaagattcgcggccctatctcgtcaccagaaattgactggtcgtccgcttacgctagaatcgctctactgaaaagctacacgatcgcgggtcctctgaactcattagcagacaaattattttcgtcattaggagaacccactccttcacagacagtgtccccccttccatgggaagtttctgagtcagagtaa

SEQ ID NO: 16 - protein, 1138 aa, synthetic construct
MKRSLWYKRFSYYLLVGLPLAFFVLLPKFLSSESGKYLFLSVLNKETGLQCEIEQLHLSWFGSQTAKKVRIRGIDSESEIFSAEQIIVSGSLPRLLLYRFPKSLTLTGWTLQIDESLSINSPSLYHLDPGSLLSKIERSDITSELGSITMKTIKGSTLSVSGFYVKKTAQQLLIRALTKENDIQGSVSVEGALSPHFLLNVQLSSVPASLFKLFIASPSIDRILSTEDLINLTAKAHQEKDSTLITLTTEGNQITAKLRGHIRDNTFLITQGGASSIVLQPAITSSILSELLAVETSIRSKEAHLFISDAKLPLSISKWNSSEFSLQANLPQISVETSDPNLSIQTENTKISVKKSDRFTLIRSSSAARLGGASPSYIHGSLSIDNRKHSAEFHIQQSLLPHTYLRALLPKPFEINIPLEVPYYTLDVKGKYRNTQLSCEAFLDNPLLRLSCTLSGPLQTLQFNGEGAYTLSERWKDRFSPYFLQMQTSFSGKMHFTQRHIFFPKLSVKLFAGENEVFIHGKFGKANEEIKPSNTSLLVYGNLFSLPMDLCSSKFAPLQMTHSSFSLHSDGGKGVLKGNLKLSITDPEFPHLSETKLLIPDIVISSLDPTSPWDAENISIQASGELLQLPVDRLIRLQHKDASLSRYIGETGKASFQLLYSPDKEETISIASTFKTDALTGDFRFVMDSSLSLTENTYGSLQWEVSPERYSSFFEKASSSPSCLLHRPTTFRLALSKISCSDKKTGYSCLSLLSEGGIEGKLSSTPLVFYDHLSKENFIVNNINGSVHAQNLNDLIQYKFTGNCLAPNQDNKSPVSFVIEGEAKNILMNETRSLTQTATWTNIPTSFITGIVPISPGVRAKLTSLAGPKIHISIYNDFYKGEGPLTIKVDSENLTGYLPLELTEHAILLKEDLTASLHINEEINKAFLREFNPLISEGGAYSKHPVSLRVNPQNFYLPIKPYSFENFRIQSASLDFGKIEIENTGTMQDLFQFLDVEAEQQRVESWFTPIFFSVQNGQIIYKRFDALIDGRIRLALWGKTDVIRDRLAMTLGIDPELIKKLFRNTMLKTKNFFLIKIRGPISSPEIDWSSAYARIALLKSYTIAGPLNSLADKLFSSLGEPTPSQTVSPLPWEVSESE

SEQ ID NO: 17 - DNA, 1098 bp, synthetic construct
  source 1..1098: mol_type = other DNA
atggtaagtttcgatttaagtgtgacaacaacgaatattggtgcaggatatgacgatattcaacgtatgttaaacggtgtgacttgttcttccggaggaatgggattattaactccttccgcatgttctccaatgagtagtttctgttcttctaaccagccctatagtgctagagatttaaagaatcgcattcaccagttttgtcagcattcgggtcctattacgggattttattccttgtacaatgagaagattatgtttgaggaggctcttcttgttccgactgttcttgaagctgtagagtcaactttttggatatcagctctttctcgtttaggaggagagcgtccttcgacttttgacacggtaattctgagcttttttgttgggcttatttctttggtgtgcggggcgatgtttgttggtattgtttctagtgccgtgaaaatttatcgtcttatgcagacaatgaggcaagcacgtactttgaatgaaaatgtgcaaagactattagctccacaagctacgaacatgcggtctgcttttgcaaaacttaaagggattgttgcttcgaaagcgcttgatcaagtagagcaaggttatcgcaaattcagaaatcgaatgattacttcttttgttgctaatgctctaattacaatagccttttgtgctttattagcatctgtaattctttctgctttcttcattggtggagcaagtgggtgtctgatggcagcgttcttcggatgtttaggagtgggacttggatctttgactattggaatgcttgtggggattgtctccgctatctgtcagcgtaagcataaacaagaagctgcacgatgcattcagagaggtattttttactcccttattttggagcaaatgcaaagattccccaaagacttttttagagatcctgtggcgaaaagtattatggccattcaagcaggagaagctttggatgaaggaaaattgtcttggaaagaaatgccaagcattacagcttgcttaggaagagagggattagatgctcaagcctattcctttatttcttcttctcccttggatggtcgtatagaagaagcttttcgatga

SEQ ID NO: 18 - protein, 365 aa, synthetic construct
MVSFDLSVTTTNIGAGYDDIQRMLNGVTCSSGGMGLLTPSACSPMSSFCSSNQPYSARDLKNRIHQFCQHSGPITGFYSLYNEKIMFEEALLVPTVLEAVESTFWISALSRLGGERPSTFDTVILSFFVGLISLVCGAMFVGIVSSAVKIYRLMQTMRQARTLNENVQRLLAPQATNMRSAFAKLKGIVASKALDQVEQGYRKFRNRMITSFVANALITIAFCALLASVILSAFFIGGASGCLMAAFFGCLGVGLGSLTIGMLVGIVSAICQRKHKQEAARCIQRGIFYSLILEQMQRFPKDFFRDPVAKSIMAIQAGEALDEGKLSWKEMPSITACLGREGLDAQAYSFISSSPLDGRIEEAFR

SEQ ID NO: 19 - DNA, 1032 bp, synthetic construct
  source 1..1032: mol_type = other DNA
atgttaaacggtgtgacttgttcttccggaggaatgggattattaactccttccgcatgttctccaatgagtagtttctgttcttctaaccagccctatagtgctagagatttaaagaatcgcattcaccagttttgtcagcattcgggtcctattacgggattttattccttgtacaatgagaagattatgtttgaggaggctcttcttgttccgactgttcttgaagctgtagagtcaactttttggatatcagctctttctcgtttaggaggagagcgtccttcgacttttgacacggtaattctgagcttttttgttgggcttatttctttggtgtgcggggcgatgtttgttggtattgtttctagtgccgtgaaaatttatcgtcttatgcagacaatgaggcaagcacgtactttgaatgaaaatgtgcaaagactattagctccacaagctacgaacatgcggtctgcttttgcaaaacttaaagggattgttgcttcgaaagcgcttgatcaagtagagcaaggttatcgcaaattcagaaatcgaatgattacttcttttgttgctaatgctctaattacaatagccttttgtgctttattagcatctgtaattctttctgctttcttcattggtggagcaagtgggtgtctgatggcagcgttcttcggatgtttaggagtgggacttggatctttgactattggaatgcttgtggggattgtctccgctatctgtcagcgtaagcataaacaagaagctgcacgatgcattcagagaggtattttttactcccttattttggagcaaatgcaaagattccccaaagacttttttagagatcctgtggcgaaaagtattatggccattcaagcaggagaagctttggatgaaggaaaattgtcttggaaagaaatgccaagcattacagcttgcttaggaagagagggattagatgctcaagcctattcctttatttcttcttctcccttggatggtcgtatagaagaagcttttcgatga

SEQ ID NO: 20 - protein, 343 aa, synthetic construct
MLNGVTCSSGGMGLLTPSACSPMSSFCSSNQPYSARDLKNRIHQFCQHSGPITGFYSLYNEKIMFEEALLVPTVLEAVESTFWISALSRLGGERPSTFDTVILSFFVGLISLVCGAMFVGIVSSAVKIYRLMQTMRQARTLNENVQRLLAPQATNMRSAFAKLKGIVASKALDQVEQGYRKFRNRMITSFVANALITIAFCALLASVILSAFFIGGASGCLMAAFFGCLGVGLGSLTIGMLVGIVSAICQRKHKQEAARCIQRGIFYSLILEQMQRFPKDFFRDPVAKSIMAIQAGEALDEGKLSWKEMPSITACLGREGLDAQAYSFISSSPLDGRIEEAFR

SEQ ID NO: 21 - DNA, 1866 bp, synthetic construct
  source 1..1866: mol_type = other DNA
atgcgcagatctgtttgttacgttactccttcagttgctagggctggtcaaatttctacctggaggtttgaatattcttcagctaatttccttcccgaggggacattattaaaatttgatttaggaatagatggacgtcctatagactgggagattccttctacagacctctctcaaccatgtaatacgatctacttggaaactccctctgagagtatcgtaacagcaaaagctgtttatgctcccggatcctacatccctacatttgaatttattcttccttgcgaaatagaagcaggagacaccttctccatcattcttggaagttctcctaattttcctcaagaagatgcttctggcaacggagcccaattatttacccagcgtcgtaaacctttttccctttacgttgatccaacgggaaaaggaaattttgaagatcctgatatcttcacaatagacatccgcggaaatgttttaaaaaatatccgtatctttgctccttcttatgtggtcaaaaataagcgctttgatattacagttcgcttcgaagatgaatttggaaatttaacaaacttttccccagaagagacccagatcgaactttcgtatgagcatctgcgagaaaatctcagttggcaattatttattcctgaaacaggattcgttattctcccgaatctttatttcaatgaacctgggatttaccgtattcaattacgcaatcaagccaccaaagagatttttacgtctgctccaatcaaatgttttacagaaacgtcaccccatcttttatggggtctgctgcatggagaatctgagcgtgtagattcggaaggcaatattgaatcctgcttacgttatttccgagatgactgcgctttgaacttctttgccacatcttctttcgaaattcaagatgggcttactcccgaaacgattaaaagcattaatcaaacggtctctgatttcaatgaagaagatcgtttcattgccctgtcgggggcacaatatgtttctgaagaacctggcgagggaattcgtgaagtccttctaataaaagagcctaaatctccaggaaagcacaaagagtgtaagttattccccctatctaaactatataagcaatcaaccagtcatgagttaatttcgatccctagctttactgcatcgaaaaaatttggttgtgattttcaaaatttccatgccgaatttgagcgagttgtagagatttacaatgcctggggatgttctgaaagaactgaagctgaagggaatccattccctattaaaggttctatcgactcagagaatccagaagggactattttgtctgctttaaagagaaatctacgttttggatttgtagctggcggtctcgatgatagaaacttgtacagtaacttttttgattccgatcaacagcaatactctcctggattaactgcagtcatttgtaataaatattctcgtgattctctgcttgaagctctataccaacgacaatgctatgcgacaacaggtcaaagaatcattgtaagttttcaaattacatcggctcctatgggttctgagctctccacagctatcaaaccaggactaatgattaatagacatatttcaggatacgtagcggggactgcaaagatttctactatcgaaatcatccgtaatggagatacactccacaccttctaccctgatggcaacaattttgaatatgagtatgacgatctgactccatttgctcaggctactcttgcagatcctaaaaatggggctccttttgccttttattacttacgagtaactcaagaaaatggagctatggcatggagttctcctatttggatagatctcaactaa

SEQ ID NO: 22 - protein, 621 aa, synthetic construct
MRRSVCYVTPSVARAGQISTWRFEYSSANFLPEGTLLKFDLGIDGRPIDWEIPSTDLSQPCNTIYLETPSESIVTAKAVYAPGSYIPTFEFILPCEIEAGDTFSIILGSSPNFPQEDASGNGAQLFTQRRKPFSLYVDPTGKGNFEDPDIFTIDIRGNVLKNIRIFAPSYVVKNKRFDITVRFEDEFGNLTNFSPEETQIELSYEHLRENLSWQLFIPETGFVILPNLYFNEPGIYRIQLRNQATKEIFTSAPIKCFTETSPHLLWGLLHGESERVDSEGNIESCLRYFRDDCALNFFATSSFEIQDGLTPETIKSINQTVSDFNEEDRFIALSGAQYVSEEPGEGIREVLLIKEPKSPGKHKECKLFPLSKLYKQSTSHELISIPSFTASKKFGCDFQNFHAEFERVVEIYNAWGCSERTEAEGNPFPIKGSIDSENPEGTILSALKRNLRFGFVAGGLDDRNLYSNFFDSDQQQYSPGLTAVICNKYSRDSLLEALYQRQCYATTGQRIIVSFQITSAPMGSELSTAIKPGLMINRHISGYVAGTAKISTIEIIRNGDTLHTFYPDGNNFEYEYDDLTPFAQATLADPKNGAPFAFYYLRVTQENGAMAWSSPIWIDLN

SEQ ID NO: 23 - DNA, 1865 bp, synthetic construct
  source 1..1865: mol_type = other DNA
atcgcagatctgtttgttacgttactccttcagttgctagggctggtcaaatttctacctggaggtttgaatattcttcagctaatttccttcccgaggggacattattaaaatttgatttaggaatagatggacgtcctatagactgggagattccttctacagacctctctcaaccatgtaatacgatctacttggaaactccctctgagagtatcgtaacagcaaaagctgtttatgctcccggatcctacatccctacatttgaatttattcttccttgcgaaatagaagcaggagacaccttctccatcattcttggaagttctcctaattttcctcaagaagatgcttctggcaacggagcccaattatttacccagcgtcgtaaacctttttccctttacgttgatccaacgggaaaaggaaattttgaagatcctgatatcttcacaatagacatccgcggaaatgttttaaaaaatatccgtatctttgctccttcttatgtggtcaaaaataagcgctttgatattacagttcgcttcgaagatgaatttggaaatttaacaaacttttccccagaagagacccagatcgaactttcgtatgagcatctgcgagaaaatctcagttggcaattatttattcctgaaacaggattcgttattctcccgaatctttatttcaatgaacctgggatttaccgtattcaattacgcaatcaagccaccaaagagatttttacgtctgctccaatcaaatgttttacagaaacgtcaccccatcttttatggggtctgctgcatggagaatctgagcgtgtagattcggaaggcaatattgaatcctgcttacgttatttccgagatgactgcgctttgaacttctttgccacatcttctttcgaaattcaagatgggcttactcccgaaacgattaaaagcattaatcaaacggtctctgatttcaatgaagaagatcgtttcattgccctgtcgggggcacaatatgtttctgaagaacctggcgagggaattcgtgaagtccttctaataaaagagcctaaatctccaggaaagcacaaagagtgtaagttattccccctatctaaactatataagcaatcaaccagtcatgagttaatttcgatccctagctttactgcatcgaaaaaatttggttgtgattttcaaaatttccatgccgaatttgagcgagttgtagagatttacaatgcctggggatgttctgaaagaactgaagctgaagggaatccattccctattaaaggttctatcgactcagagaatccagaagggactattttgtctgctttaaagagaaatctacgttttggatttgtagctggcggtctcgatgatagaaacttgtacagtaacttttttgattccgatcaacagcaatactctcctggattaactgcagtcatttgtaataaatattctcgtgattctctgcttgaagctctataccaacgacaatgctatgcgacaacaggtcaaagaatcattgtaagttttcaaattacatcggctcctatgggttctgagctctccacagctatcaaaccaggactaatgattaatagacatatttcaggatacgtagcggggactgcaaagatttctactatcgaaatcatccgtaatggagatacactccacaccttctaccctgatggcaacaattttgaatatgagtatgacgatctgactccatttgctcaggctactcttgcagatcctaaaaatggggctccttttgccttttattacttacgagtaactcaagaaaatggagctatggcatggagttctcctatttggatagatctcaactaa